(19) United States
(12) Patent Application Publication
(10) Pub. No.: US 2023/0303642 A1
(43) Pub. Date: Sep. 28, 2023

(54) Methods and Compositions for Modulating Toll-Like Receptor 7 (TLR7) Function

(71) Applicant: The Regents of the University of California, Oakland, CA (US)

(72) Inventors: Gregory M. Barton, Piedmont, CA (US); Nicholas Anton Lind, Oakland, CA (US); Olivia Majer, Berlin (DE); Bo Liu, Richmond, CA (US)

(21) Appl. No.: 18/000,461

(22) PCT Filed: Jun. 6, 2021
(86) PCT No.: PCT/US2021/036060

§ 371 Date: Dec. 1, 2022

Related U.S. Application Data
(60) Provisional application No. 63/038,575, filed on Jun. 12, 2020.

(51) Int. Cl. C07K 14/47; C12N 15/85; A61K 48/00

(52) U.S. Cl. CPC C07K 14/4702; A61K 48/005; C12N 15/85

(57) ABSTRACT

Disclosed herein are Unc93bl mutations that modulated the trafficking and/or signaling of TLR7, and compositions and methods of using thereof.

### CROSS-REFERENCE TO RELATED APPLICATIONS

This application claims the benefit of U.S. Patent Application No. 63/038,575, filed Jun. 12, 2020, which is herein incorporated by reference in its entirety.

### REFERENCE TO A SEQUENCE LISTING SUBMITTED VIA EFS-WEB

The content of the ASCII text file of the sequence listing named “20210606_034044_205WO1_ST25” which is 32.3 kb in size was created on Jun. 6, 2021 and electronically submitted via EFS-Web herewith the application is incorporated herein by reference in its entirety.

### BACKGROUND OF THE INVENTION

### 1. Field of the Invention

The field of the invention relates to methods and compositions for modulating trafficking and signaling of Toll-Like Receptor 7 (TLR7) and Toll-Like Receptor 8 (TLR8).

### 2. Description of the Related Art

Toll-Like Receptors (TLRs) play important roles in the recognition of self and non-self antigens, the detection of invading pathogens, innate and adaptive immunity, and regulation of cytokine production, proliferation, and survival. TLRs recognize pathogen-associated molecular patterns (PAMPs), which are expressed on infectious agents, or damage-associated molecular patterns (DAMPs), which are endogenous molecules released from necrotic or dying cells. Stimulation of TLRs initiates signaling cascades that lead to the activation of various transcription factors, such as AP-1, NF-κB, and interferon regulatory factors (IRFs). Signaling by TLRs results in a variety of cellular responses such as the production of interferons (IFNs), pro-inflammatory cytokines, and effector cytokines. TLRs are located on the plasma membrane with the exception of TLR3, TLR7, TLR8, and TLR9 which are localized in the endosomal compartment.

There are two primary TLR signaling pathways: The myeloid differentiation primary response protein 88 (MyD88) pathway, and the TIR domain-containing adaptor-inducing IFNβ (TRIF) pathway. The MyD88 pathway is common to all the TLRs except TLR3. TLR activation and dimerization results in the recruitment of adaptor proteins via the cytoplasmic TIR domain. Adaptor proteins include the TIR-domain containing proteins, MyD88, TIRAP (TIR-associated protein), Mal (MyD88 adaptor-like protein), TRIF (TIR domain-containing adaptor protein-inducing IFN-β), and TRAM (TRIF-related adaptor molecule).

The different functional roles of TLRs are the result, in part, of the different signaling responses caused by different adaptor molecules. For example, TLR4 and TLR2 signaling requires the adaptor TIRAP/Mal and TLR3 triggers the production of IFN-β in response to double-stranded RNA through the adaptor TRIF/TICAM-1. As another example, recruitment of MyD88 recruits IRAK1 and IRAK4. IRAK4 subsequently activates IRAK1 by phosphorylation. Both IRAK1 and IRAK4 temporarily associate with TRAF6 thereby leading to its ubiquitination. Following ubiquitination, TRAF6 forms a complex with TAB2/TAB3/TAK1 which thereby induces TAK1 activation. TAK1 then couples to the IKK complex which leads to the phosphorylation of IκB and the subsequent nuclear localization of NF-κB. Activation of NF-κB triggers the production of pro-inflammatory cytokines such as TNF-α, IL-1 and IL-12.

The TRIF-dependent pathway is believed to be specific for only few TLRs, such as TLR3 and TLR4. Transcription factors, including NF-κB, activating protein-1 (AP-1), and interferon (IFN) regulatory factor (IRF) family members, may be activated by the TRIF-dependent pathway, and thereby induce the production of pro-inflammatory cytokines and/or type I IFN (IFN1). TLR3 is activated by recognizing double-stranded RNA (dsRNA), which is followed by the recruitment of TRIF. TRIF activates TANK-binding kinase 1 (TBK1) and receptor-interacting serine/threonine kinase 1 (RIPK1). The TRIF/TBK1 signaling complex phosphorylates IRF3, allowing its translocation to the nucleus and the production of IFN1. Activation of RIPK1 causes a series of other signal transduction events. TLR4 functions as an LPS receptor in mammals, and the TLR4-myeloid differentiation protein 2 (MD2)-LPS complex activates early-phase NF-κB and mitogen-activated protein kinase (MAPK) after the recruitment of MyD88 and MyD88-adapter-like (MAL) adaptors. After entering the cell, the TLR4-MD2-LPS complex interacts with the TRIF and TIR domain-containing adapter molecule 2 (TICAM2) adaptors. The TRIF pathway induces the production of IFN1 and also activates IRF7 and late-phase NF-κB, which ultimately leads to the regulation of genes involved in the inflammatory response.

Although TLRs are highly conserved and share some structural and functional similarities, they exhibit different patterns of expression and biological roles. TLR3, TLR7, TLR8, and TLR9 recognize viral nucleic acids and induce type I IFNs. The signaling mechanisms leading to the induction of type I IFNs differ depending on the given TLR and interferon regulatory factors (IRFs). IRF3, IRF5 and IRF7 are direct transducers of virus-mediated TLR signaling. TLR3 and TLR4 activate IRF3 and IRF7, while TLR7 and TLR8 activate IRF5 and IRF7.

TLR7 recognizes single-stranded RNA in endosomes, which is a common feature of viruses such as HIV, influenza, and HCV. TLR7 recognizes single-stranded RNA of viruses such as HIV and HCV. TLR7 recognizes GU-rich single-stranded RNA; however, the presence of GU-rich sequences in the single-stranded RNA is insufficient to stimulate TLR7. TLR7 is involved in the pathogenesis of autoimmune disorders such as Systemic Lupus Erythematosus (SLE) and the regulation of antiviral immunity.

### SUMMARY OF THE INVENTION

In some embodiments, the present invention is directed to a mutant Unc93b1 protein comprising at least one amino acid mutation as compared to its unmutated wildtype sequence, with the proviso that the at least one amino acid mutation does not correspond to D34A; Y99A; Y154A; K197A; H412R; PRQ(524,525,526)/AAA; PKP(530,531,532)/AAA; DNS(545,546,547)/AAA; S547A; DES(548,549,550)/AAA of SEQ ID NO: 1. In some embodiments, the at least one amino acid mutation is selected from Group A, Group B, Group C, Group D, and Group E mutations described herein. In some embodiments, the at least one amino acid mutation corresponds to one or more mutations as set forth in FIG. 1. In some embodiments, the unmutated wildtype sequence comprises 90%, 91%, 92%, 93%, 94%, 95%, 96%, 97%, 98%, 99%, or 100% sequence identity to SEQ ID NO: 1. In some embodiments, the unmutated wildtype sequence comprises at least 90%, at least 91%, at least 92%, at least 93%, at least 94%, at least 95%, at least 96%, at least 97%, at least 98%, at least 99%, or 100% sequence identity to SEQ ID NO: 2. In some embodiments, the at least one amino acid mutation corresponds to one of the mutations provided in FIG. 1. In some embodiments, the amino acid sequence of the mutant Unc93b1 protein comprises less than 100% sequence identity to naturally occurring unc-93 homolog B1 proteins. In some embodiments, the amino acid sequence of the mutant Unc93b1 protein comprises 85%, 86%, 87%, 88%, 89%, 90%, 91%, 92%, 93%, 94%, 95%, 96%, 97%, 98%, or 99% sequence identity to SEQ ID NO: 1. In some embodiments, the amino acid sequence of the mutant Unc93b1 protein comprises 85%, 86%, 87%, 88%, 89%, 90%, 91%, 92%, 93%, 94%, 95%, 96%, 97%, 98%, or 99% sequence identity to SEQ ID NO: 2.

In some embodiments, the present invention is directed to a method of modulating the trafficking and/or signaling of a Toll-Like Receptor in a cell or subject, which comprises administering to the cell or subject one or more mutant Unc93b1 proteins as described herein, e.g., as described in the above paragraph. In some embodiments, the Toll-Like Receptor is Toll-Like Receptor 7 (TLR7) or a Toll-Like Receptor 8 (TLR8). In some embodiments, when compared to a negative control, the signaling of the Toll-Like Receptor is increased and the at least one amino acid mutation corresponds to one or more of the following mutations of SEQ ID NO: 1: EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; KPK(531,532,535); QHK(533,534,535); VRG(536,537,538); LEE(542,543,544); DME(551,552,553); and S(187,212,432,547,550). In some embodiments, when compared to a negative control, the signaling of the Toll-Like Receptor is decreased and the at least one amino acid mutation corresponds to one or more of the following mutations of SEQ ID NO: 1: EVE(2,3,4); PP(6,9); Y8; VG(10,12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); PP(524,527); PRI(527,528,529); PP(527,530); Y539; Y541; GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and RR(50,51) + RRR(54,55,57) + RR(339,341). In some embodiments, when compared to a negative control, the trafficking of the Toll-Like Receptor is decreased and the at least one amino acid mutation corresponds to one or more of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and RR(50,51) + RRR(54,55,57) + RR(339,341). In some embodiments, a nucleic acid molecule encoding the one or more mutant Unc93b1 proteins is administered to the cell or subject. In some embodiments, a host cell that expresses the one or more mutant Unc93b1 proteins is administered to the subject. In some embodiments, the one or more mutant Unc93b1 proteins is administered by modifying a Unc93b1 gene of the cell or subject to express the one or more mutant Unc93b1 proteins, wherein the Unc93b1 gene is endogenous to the cell or subject. In some embodiments, the one or more mutant Unc93b1 proteins is administered in the form of a pharmaceutical composition. In some embodiments, the subject is in need of toll-like receptor modulation.

In some embodiments, the present invention is directed to a nucleic acid molecule that encodes a mutant Unc93b1 protein as described herein.

In some embodiments, the present invention is directed to a host cell comprising a mutant Unc93b1 protein as described herein or a nucleic acid molecule that encodes the mutant Unc93b1 protein.

In some embodiments, the present invention is directed to a composition comprising (a) a mutant Unc93b1 protein, a nucleic acid molecule, and/or the host cell as described herein, and (b) a pharmaceutically acceptable carrier.

In some embodiments, the present invention is directed to a kit comprising (a) a mutant Unc93b1 protein, a nucleic acid molecule, a host cell, and/or a composition as described herein, (b) packaged together with a drug delivery device.

In some embodiments, the methods described herein are used for inducing or increasing regulatory T cell proliferation, production of amphiregulin, and/or tissue repair in a subject.

In some embodiments, the methods described herein are used for inducing or increasing CD8+ T cell proliferation, production of interferon-gamma, and/or a costimulatory immune response in a subject.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 is a table summarizing the impact various Unc93b1 mutations exert on TLR7 trafficking and signaling. The Unc93b1 protein having the indicated mutations is SEQ ID NO: 1 (Accession No. Q8VCW4.2) and the TLR7 protein is Accession No. XP_006528776.1.

FIG. 2 is a sequence alignment between human (SEQ ID NO: 2, Accession No. NP_112192.2) and mouse (SEQ ID NO: 1, Accession No. Q8VCW4.2) Unc93b1 protein sequences.

FIG. 3 is a sequence alignment of highly conserved regions of human, zebrafish, rock pigeon, western claw frog, and elephant shark Unc93b1 proteins. The sequence identifiers of the Unc93b1 proteins are SEQ ID NO: 2 (human), SEQ ID NO: 3 (zebrafish), SEQ ID NO: 4 (rock pigeon), SEQ ID NO: 5 (western claw frog), and SEQ ID NO: 6 (elephant shark).

FIG. 4 and FIG. 5: TLR7 stimulus causes Regulatory T cells to divide and produce amphiregulin, an important mediator of tissue repair. FIG. 4: Congenically-marked wildtype and TLR7^{KO} CD4⁺ T cells were positively enriched on a Miltenyi magnetic column, mixed at a 1:1 ratio (10⁵ per genotype), and subjected to a 3 day stimulation with the noted conditions. R848 is a ligand specific for TLR7, and was provided at a concentration of 1 µg/ml. anti-CD3 antibody provides stimulation through the T cell receptor, and was provided at a concentration of 1 µg/ml. All samples also received 150 U/ml of IL-2 cytokine to improve viability. After 3 days, the ratio of wildtype to TLR7^{KO} regulatory T cells was assayed by flow cytometry using antibodies against the congenic markers and Foxp3. FIG. 5: Identical experimental setup as in FIG. 4. After a 3-day stimulation, samples were subjected to intracellular antibody staining for amphiregulin, the primary molecular mediator of tissue repair by regulatory T cells. Amphiregulin staining was measured by flow cytometry using anti-mAmphiregulin biotinylated antibody, R&D Cat. # BAF989.

FIG. 6 and FIG. 7: TLR7 stimulus causes CD8⁺ T cells to divide and produce critical immune cytokine interferon gamma. TLR7 can also synergize with T cell receptor signaling to achieve an even greater, “costimulatory” effect. FIG. 6: Congenically-marked wildtype and TLR7^{KO} CD8+ T cells were positively enriched on a Miltenyi magnetic column, mixed at a 1:1 ratio (10⁵ per genotype), and subjected to a 3-day stimulation with the noted conditions. R848 is a ligand specific for TLR7, and was provided at a concentration of 1 µg/ml. anti-CD3 antibody provides stimulation through the T cell receptor, and was provided at a concentration of 1 µg/ml. All samples also received 150 U/ml of IL-2 cytokine to improve viability. After 3 days, the ratio of wildtype to TLR7^{KO} CD8⁺ T cells was assayed by flow cytometry using antibodies against the congenic markers. FIG. 7 Identical experimental setup as FIG. 6. After a 3-day stimulation, samples were “restimulated” for 4 hours with PMA/ionomycin and golgi plug to assay antibody production. Following restimulation, samples were subjected to intracellular antibody staining for interferon gamma (Ifn-g), one of the classic effector cytokines produced by CD8⁺ T cells. Ifn-g staining was measured by flow cytometry using PE rat anti-mouse IFN-g antibody, BD Pharmingen Cat # 562020.

### DETAILED DESCRIPTION OF THE INVENTION

Unc-93 homolog B1 (Unc93b1) is a twelve-pass transmembrane protein that binds a subset of TLRs (TLR3, TLR5, TLR7, TLR8, TLR9, TLR11, TLR12, and TLR13) in the endoplasmic reticulum (ER) and facilitates their trafficking to endosomes. As disclosed herein, Toll-Like Receptor 7 (TLR7) trafficking and signaling are differentially modulated by different Unc93b1 mutations.

### Influence of Unc93bl Mutations on TLR Trafficking and Signaling

A library of mutant Unc93b1 genes was generated and then each mutant was stably expressed in a RAW macrophage cell line in which both endogenous Unc93bl alleles were disrupted by Cas9 genome editing and the effect of each mutation on TLR trafficking and signaling was evaluated using methods in the art and as disclosed herein.

Deletion of endogenous Unc93b1 led to lack of responses to nucleic acids and failure of TLR7 to traffic to endosomes. Each mutant Unc93b1 cell line was stimulated with ligands for TLR3, TLR4, TLR5, TLR7, TLR9, and TLR13, and then the trafficking and signaling of each TLR was assayed using methods in the art. For example, the levels of the cleaved forms of the TLRs in a mutant Unc93b1 cell line was measured and compared to that of Unc93b1^{WT} control cells to determine whether the given Unc93b1 mutation had an effect on TLR trafficking. Similarly, activation of the MAPK and NFκB signaling pathways and assembly of Myddosome complexes in a mutant Unc93b1 cell line were measured and compared to that of Unc93b1^{WT} control cells to determine whether the given Unc93b1 mutation had an effect on TLR signaling.

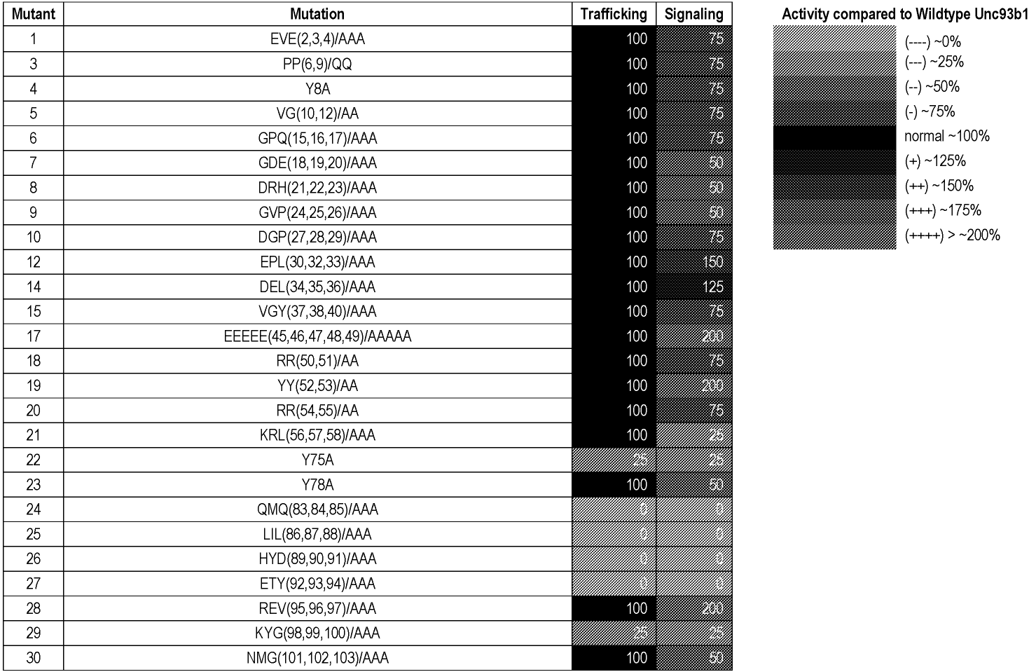

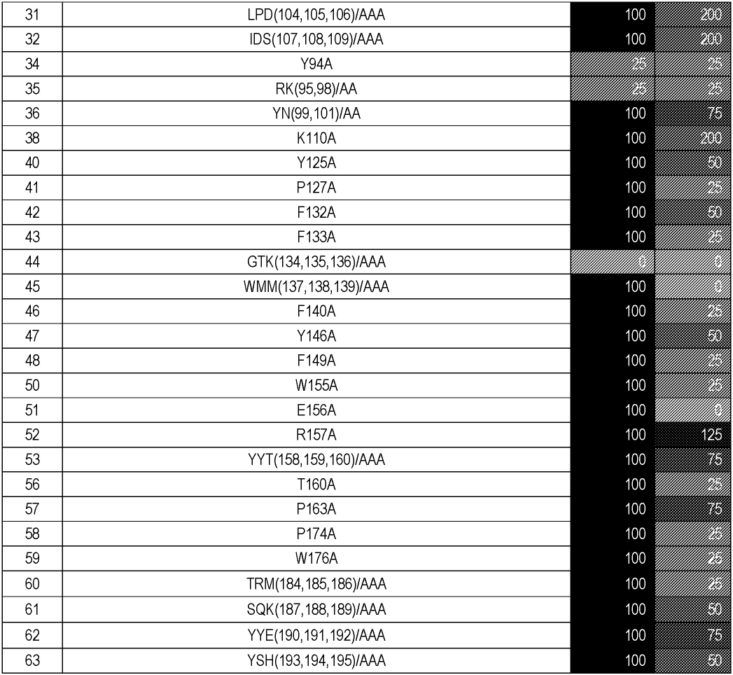

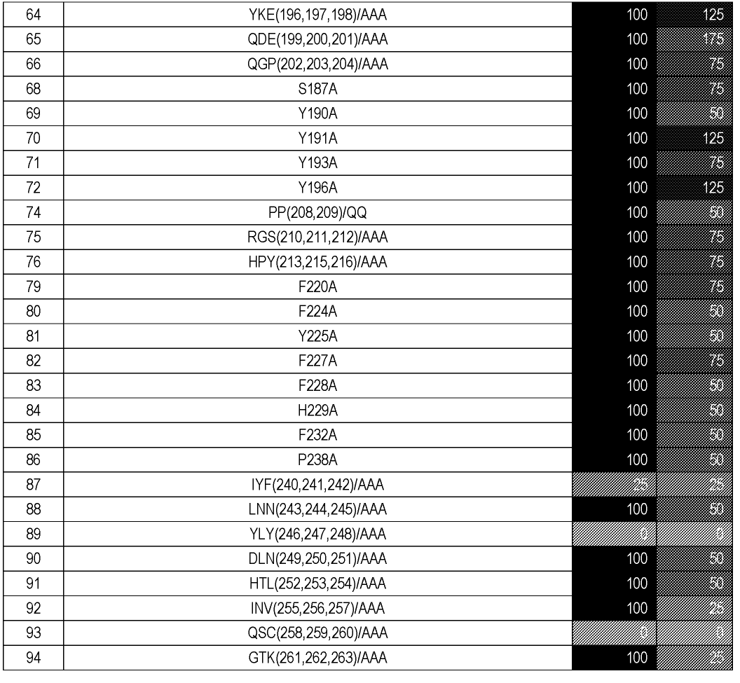

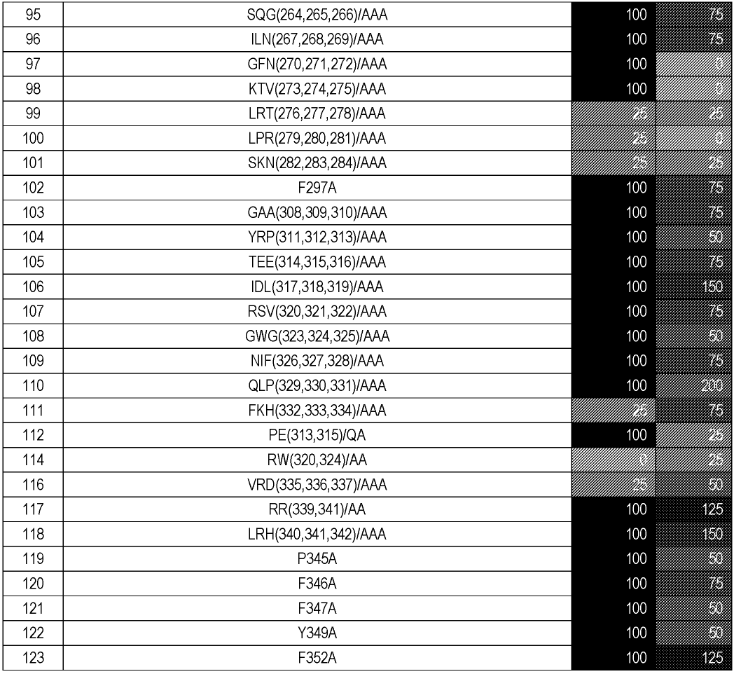

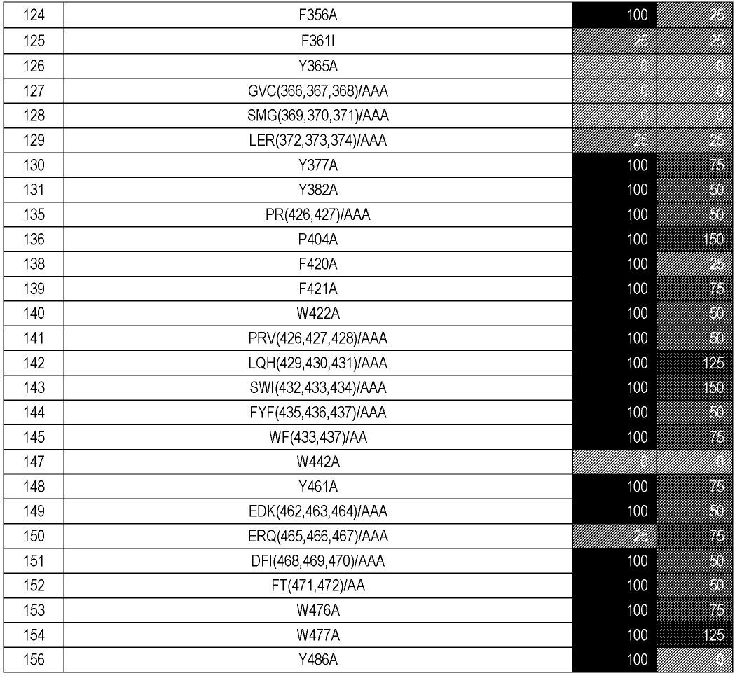

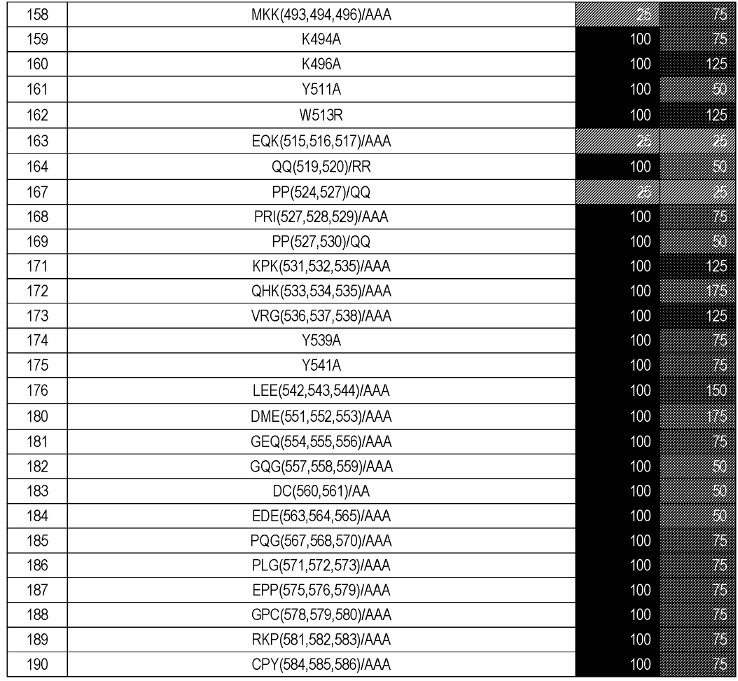

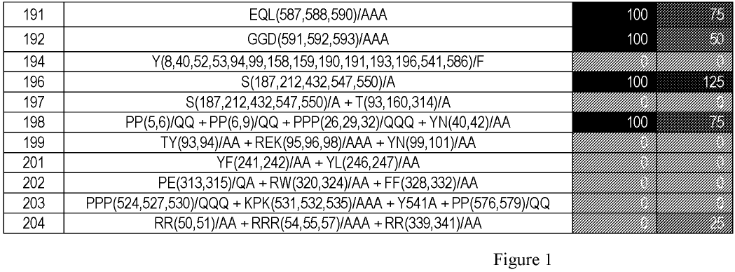

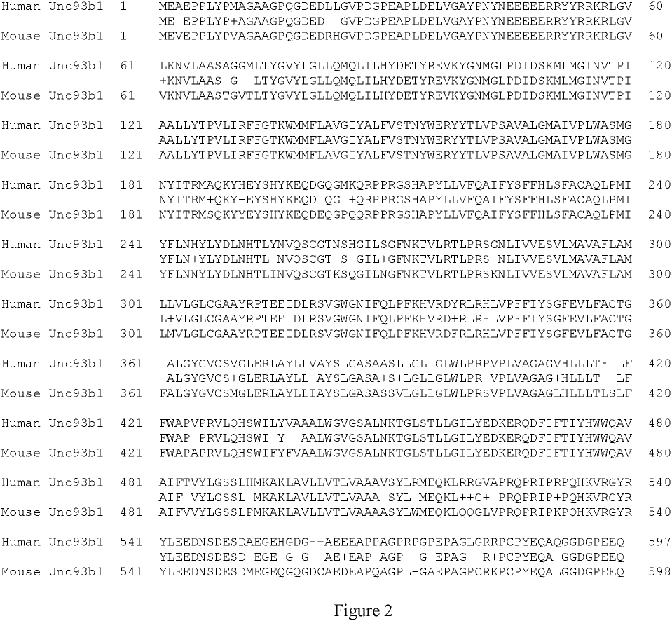

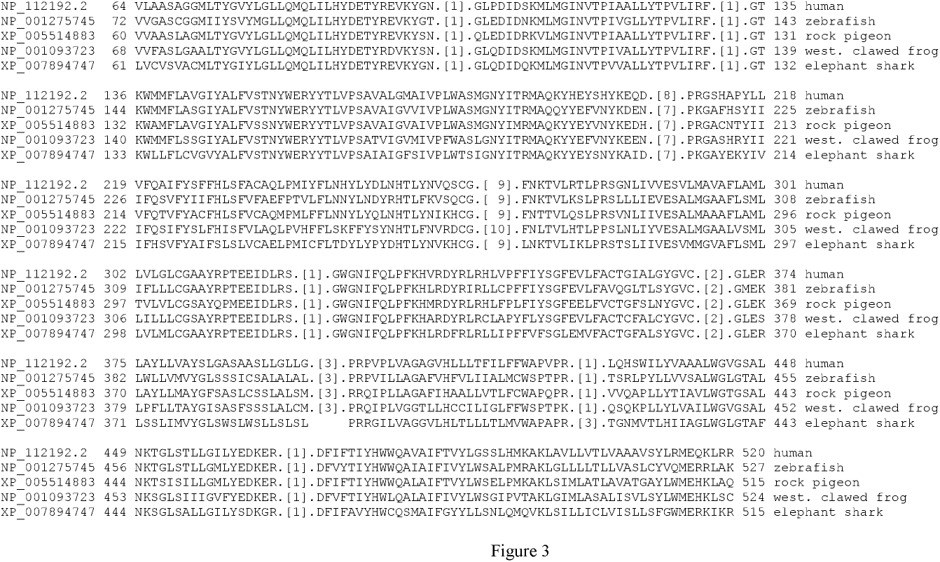

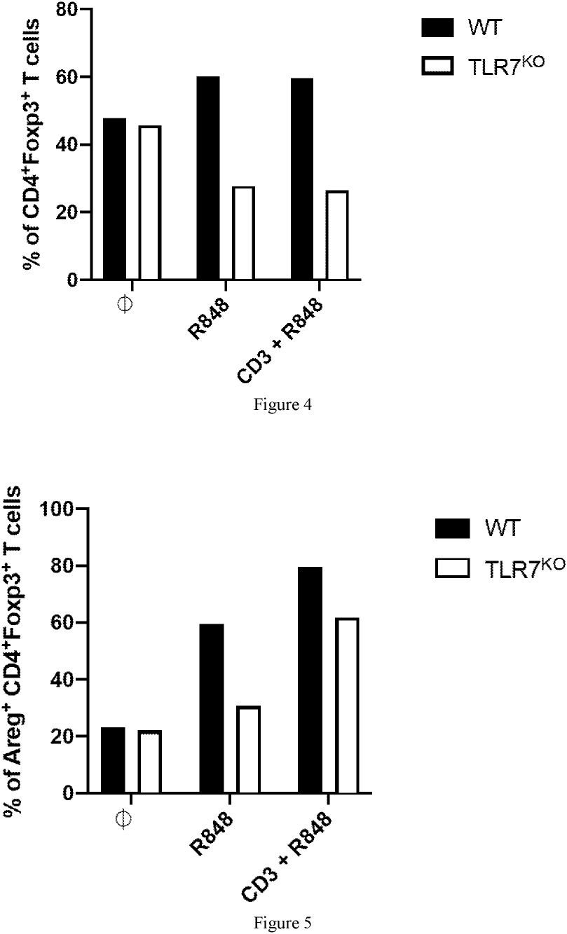

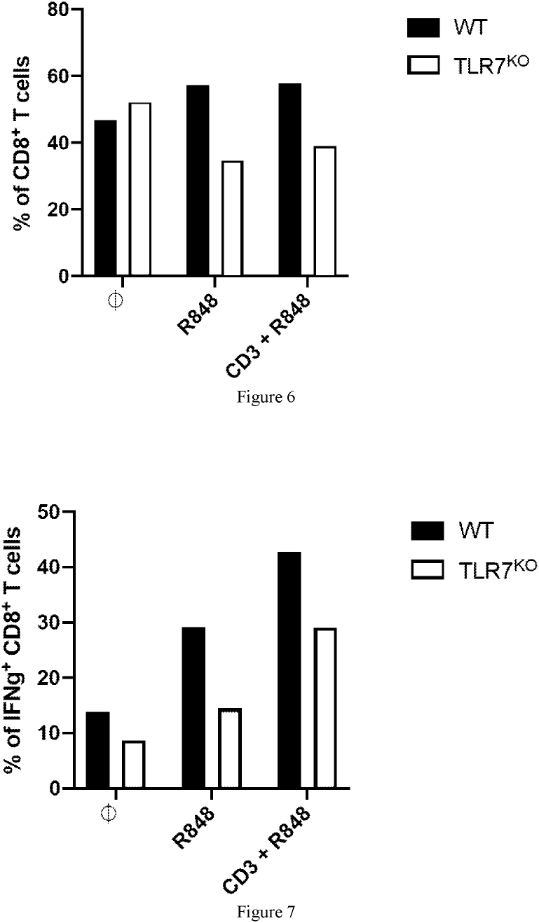

FIG. 1 is a table which provides the Unc93b1 mutations of each mutant and the amount each mutation increased or decreased TLR7 trafficking and signaling compared to Unc93b1^{WT}. FIG. 2 is a sequence alignment showing that human and mouse Unc93b1 have 90% sequence identity. A plurality of regions from amino acid residues 64 to 520 of human Unc93b1 are highly conserved across a variety of diverse species including the zebrafish, rock pigeon, western clawed frog, and elephant shark. See FIG. 3. Because of the highly conserved regions Unc93b1, it is believed that Unc93b1 homologs, orthologs, and paralogs that have one or more amino acid mutations that correspond to those provided in FIG. 1 will similarly modulate the trafficking and signaling of the TLR7 to which the given Unc93b1 homolog, ortholog, and paralog is natively associated. Particularly, because of the high sequence identity between human Unc93b1 and mouse Unc93b1, it is believed that mutations in human Unc93b1 that correspond to those provided in FIG. 1 will similarly modulate the trafficking and signaling of human TLR7.

In fact, human Unc93b1 allelic variants encoding mutations corresponding to P532T, Y539D, D545V, and D545Y in mouse Unc93b1 were expressed in HEK293T cells along with human TLR7 to determine their impact on human TLR7 trafficking and signaling. Three of the variants (Unc93b1^{Y539D}, Unc93b1^{D545V}, and Unc93b1^{D545Y}) increased TLR7 responses relative to Unc93b1^{WT}, although Unc93b1^{Y539D}, and to a lesser extent Unc93b1^{D545Y}, also increased TLR5 responses. These results indicate that mutations in human Unc93b1 at amino acid positions corresponding to mutations in mouse Unc93b1 as provided in FIG. 1 will similarly modulate the trafficking and signaling of human TLR7 as do the mouse Unc93b1 mutations modulate the trafficking and signaling of mouse TLR7.

Therefore, in some embodiments, the present invention provides Unc93b1 therapeutics. As used herein, “Unc93b1 therapeutics” include mutant Unc93b1 proteins, nucleic acid molecules that encode mutant Unc93b1 proteins, expression systems that genetically modify a given Unc93b1 gene to encode mutant Unc93b1 proteins, and cells that have been genetically modified to express mutant Unc93b1 proteins, wherein the mutant Unc93b1 proteins have at least one amino acid mutation corresponding to one or more of the following mutations of SEQ ID NO: 1:
Group A comprising Y75; Y78; QMQ(83,84,85); LIL(86,87,88); HYD(89,90,91); ETY(92,93,94); REV(95,96,97); KYG(98,99,100); NMG(101,102,103); LPD(104,105,106); IDS(107,108,109); T93; Y94; RK(95,98); YN(99,101); K110; P119; Y125; P127; F132; F133; GTK(134,135,136); WMM(137,138,139); F140; Y146; F149; W155; E156; R157; YYT(158,159,160); Y158; Y159; T160; P163; P174; W176; TRM(184,185,186); YSH(193,194,195); YKE(196,197,198); Y190; Y193; Y196; PP(208,209); RGS(210,211,212); HPY(213,215,216); R210; S212; F220; F224; Y225; F227; F228; H229; F232; P238; IYF(240,241,242); YLY(246,247,248); DLN(249,250,251); HTL(252,253,254); QSC(258,259,260); GFN(270,271,272); KTV(273,274,275); LRT(276,277,278); F297; GAA(308,309,310); YRP(311,312,313); TEE(314,315,316); IDL(317,318,319); RSV(320,321,322); GWG(323,324,325); NIF(326,327,328); QLP(329,330,331); FKH(332,333,334); PE(313,315); T314; RW(320,324); FF(328,332); VRD(335,336,337); RR(339,341); LRH(340,341,342); P345; F346; F347; Y349; F352; F356; Y365; GVC(366,367,368); LER(372,373,374); Y377; Y382; W398; LP(399,400); R401; PR(426,427); P404; F420; F421; W422; PRV(426,427,428); LQH(429,430,431); SWI(432,433,434); S432; W442; Y461; EDK(462,463,464); ERQ(465,466,467); DFI(468,469,470); FT(471,472); W476; W477; F483; Y486; MKK(493,494,496); K494; K496; Y511; and EQK(515,516,517),
Group B comprising EVE(2,3,4); PP(5,6); PP(6,9); Y8; GPQ(15,16,17); GDE(18,19,20); GVP(24,25,26); DGP(27,28,29); PPP(26,29,32); EPL(30,32,33); DEL(34,35,36); VGY(37,38,40); YN(40,42); EEEEE(45,46,47,48,49); RR(50,51); YY(52,53); RR(54,55); KRL(56,57,58); QDE(199,200,201); QGP(202,203,204); F361; P492; W513; QQ(519,520); CPY(584,585,586); EQL(587,588,590); Y(8,40,52,53,94,99,158,159,190,191,193,196,541,586); S(187,212,432,547,550); S(187,212,432,547,550) + T(93,160,314); PP(5,6) + PP(6,9) + PPP(26,29,32) + YN(40,42); TY(93,94) + REK(95,96,98) + YN(99,101); Y191 + Y196 + PP(208,209) + S212; YF(241,242) + YL(246,247); PE(313,315) + RW(320,324) + FF(328,332); PPP(524,527,530) + KPK(531,532,535) + Y541 + PP(576,579); and RR(50,51) + RRR(54,55,57) + RR(339,341),
Group C comprising VG(10,12); SQK(187,188,189); YYE(190,191,192); QQR(205,206,207); S187; Y191; LNN(243,244,245); ILN(267,268,269); LPR(279,280,281); SKN(282,283,284); SMG(369,370,371); WF(433,437); EDE(563,564,565); EPP(575,576,579); RKP(581,582,583); and GGD(591,592,593),
Group D comprising DRH(21,22,23); INV(255,256,257); GTK(261,262,263); SQG(264,265,266); FYF(435,436,437); GEQ(554,555,556); GQG(557,558,559); DC(560,561); PQG(567,568,570); PLG(571,572,573); GPC(578,579,580), and/or
Group E comprising GLV(521,522,523); PP(524,527); PRI(527,528,529); PP(527,530); KPK(531,532,535); QHK(533,534,535); VRG(536,537,538); Y539; Y541; LEE(542,543,544); DME(551,552,553); and K(197,333,531,535,582).

In some embodiments, the at least one amino acid mutation corresponds to one or more of the mutations of Group A, Group B, Group C, and/or Group D. In some embodiments, the at least one amino acid mutation corresponds to one or more of the mutations of Group A, Group B, and/or Group C. In some embodiments, the at least one amino acid mutation corresponds to one or more of the mutations of Group A and/or Group B. In some embodiments, the at least one amino acid mutation corresponds to one or more of the mutations of Group A. In some embodiments, the at least one amino acid mutation corresponds to one or more of the following mutations of SEQ ID NO: 1: Y75A; Y78A; QMQ(83,84,85)/AAA; LIL(86,87,88)/AAA; HYD(89,90,91)/AAA; ETY(92,93,94)/AAA; REV(95,96,97)/AAA; KYG(98,99,100)/AAA; NMG(101,102,103)/AAA; LPD(104,105,106)/AAA; IDS(107,108,109)/AAA; T93A; Y94A; RK(95,98)/AA; YN(99,101)/AA; K110A; P119A; Y125A; P127A; F132A; F133A; GTK(134,135,136)/AAA; WMM(137,138,139)/AAA; F140A; Y146A; F149A; W155A; E156A; R157A; YYT(158,159,160)/AAA; Y158A; Y159A; T160A; P163A; P174A; W176A; TRM(184,185,186)/AAA; YSH(193,194,195)/AAA; YKE(196,197,198)/AAA; Y190A; Y193A; Y196A; PP(208,209)/QQ; RGS(210,211,212)/AAA; HPY(213,215,216)/AAA; R210A; S212A; F220A; F224A; Y225A; F227A; F228A; H229A; F232A; P238A; IYF(240,241,242)/AAA; YLY(246,247,248)/AAA; DLN(249,250,251)/AAA; HTL(252,253,254)/AAA; QSC(258,259,260)/AAA; GFN(270,271,272)/AAA; KTV(273,274,275)/AAA; LRT(276,277,278)/AAA; F297A; GAA(308,309,310)/AAA; YRP(311,312,313)/AAA; TEE(314,315,316)/AAA; IDL(317,318,319)/AAA; RSV(320,321,322)/AAA; GWG(323,324,325)/AAA; NIF(326,327,328)/AAA; QLP(329,330,331)/AAA; FKH(332,333,334)/AAA; PE(313,315)/QA; T314A; RW(320,324)/AA; FF(328,332)/AA; VRD(335,336,337)/AAA; RR(339,341)/AA; LRH(340,341,342)/AAA; P345A; F346A; F347A; Y349A; F352A; F356A; Y365A; GVC(366,367,368)/AAA; LER(372,373,374)/AAA; Y377A; Y382A; W398A; LP(399,400)/AA; R401A; PR(426,427)/AAA; P404A; F420A; F421A; W422A; PRV(426,427,428)/AAA; LQH(429,430,431)/AAA; SWI(432,433,434)/AAA; S432A; W442A; Y461A; EDK(462,463,464)/AAA; ERQ(465,466,467)/AAA; DFI(468,469,470)/AAA; FT(471,472)/AA; W476A; W477A; F483A; Y486A; MKK(493,494,496)/AAA; K494A; K496A; Y511A; and EQK(515,516,517)/AAA. In some embodiments, the at least one amino acid mutation corresponds to one or more of the following mutations of SEQ ID NO: 1: EVE(2,3,4); F361; P492; W513; and QQ(519,520).

In some embodiments, the present invention is directed to a method of increasing the signaling of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; KPK(531,532,535); QHK(533,534,535); VRG(536,537,538); LEE(542,543,544); DME(551,552,553); and/or S(187,212,432,547,550), preferably EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; and/or S(187,212,432,547,550), more preferably EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; and/or S(187,212,432,547,550).

In some embodiments, the present invention is directed to a method of increasing the signaling of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: EPL(30,32,33)/AAA; DEL(34,35,36)/AAA; EEEEE(45,46,47,48,49)/AAAAA; YY(52,53)/AA; REV(95,96,97)/AAA; LPD(104,105,106)/AAA; IDS(107,108,109)/AAA; K110A; R157A; YKE(196,197,198)/AAA; QDE(199,200,201)/AAA; Y191A; Y196A; IDL(317,318,319)/AAA; QLP(329,330,331)/AAA; RR(339,341)/AA; LRH(340,341,342)/AAA; F352A; P404A; LQH(429,430,431)/AAA; SWI(432,433,434)/AAA; W477A; K496A; W513R; KPK(531,532,535)/AAA; QHK(533,534,535)/AAA; VRG(536,537,538)/AAA; LEE(542,543,544)/AAA; DME(551,552,553)/AAA; and/or S(187,212,432,547,550)/A, preferably EPL(30,32,33)/AAA; DEL(34,35,36)/AAA; EEEEE(45,46,47,48,49)/AAAAA; YY(52,53)/AA; REV(95,96,97)/AAA; LPD(104,105,106)/AAA; IDS(107,108,109)/AAA; K110A; R157A; YKE(196,197,198)/AAA; QDE(199,200,201)/AAA; Y191A; Y196A; IDL(317,318,319)/AAA; QLP(329,330,331)/AAA; RR(339,341)/AA; LRH(340,341,342)/AAA; F352A; P404A; LQH(429,430,431)/AAA; SWI(432,433,434)/AAA; W477A; K496A; and/or S(187,212,432,547,550)/A.

In some embodiments, the present invention is directed to a method of decreasing the signaling of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: EVE(2,3,4); PP(6,9); Y8; VG(10, 12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); PP(524,527); PRI(527,528,529); PP(527,530); Y539; Y541; GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably EVE(2,3,4); PP(6,9); Y8; VG(10, 12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), more preferably PP(6,9); Y8; GPQ(15,16,17); GDE(18,19,20); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); YLY(246,247,248); DLN(249,250,251); HTL(252,253,254); QSC(258,259,260); GFN(270,271,272); KTV(273,274,275); LRT(276,277,278); F297; GAA(308,309,310); YRP(311,312,313); TEE(314,315,316); RSV(320,321,322); GWG(323,324,325); NIF(326,327,328); PE(313,315); RW(320,324); VRD(335,336,337); P345; F346; F347; Y349; F356; Y365; GVC(366,367,368); LER(372,373,374); Y377; Y382; PR(426,427); F420; F421; W422; PRV(426,427,428); W442; Y461; EDK(462,463,464); ERQ(465,466,467); DFI(468,469,470); FT(471,472); W476; Y486; MKK(493,494,496); K494; Y511; EQK(515,516,517); CPY(584,585,586); EQL(587,588,590); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the signaling of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: EVE(2,3,4)/AAA; PP(6,9)/QQ; Y8A; VG(10, 12)/AA; GPQ(15,16,17)/AAA; GDE(18,19,20)/AAA; DRH(21,22,23)/AAA; GVP(24,25,26)/AAA; DGP(27,28,29)/AAA; VGY(37,38,40)/AAA; RR(50,51)/AA; RR(54,55)/AA; KRL(56,57,58)/AAA; Y75A; Y78A; QMQ(83,84,85)/AAA; LIL(86,87,88)/AAA; HYD(89,90,91)/AAA; ETY(92,93,94)/AAA; KYG(98,99,100)/AAA; NMG(101,102,103)/AAA; Y94A; RK(95,98)/AA; YN(99,101)/AA; Y125A; P127A; F132A; F133A; GTK(134,135,136)/AAA; WMM(137,138,139)/AAA; F140A; Y146A; F149A; W155A; E156A; YYT(158,159,160)/AAA; T160A; P163A; P174A; W176A; TRM(184,185,186)/AAA; SQK(187,188,189)/AAA; YYE(190,191,192)/AAA; YSH(193,194,195)/AAA; QGP(202,203,204)/AAA; S187A; Y190A; Y193A; PP(208,209)/QQ; RGS(210,211,212)/AAA; HPY(213,215,216)/AAA; F220A; F224A; Y225A; F227A; F228A; H229A; F232A; P238A; IYF(240,241,242)/AAA; LNN(243,244,245)/AAA; YLY(246,247,248)/AAA; DLN(249,250,251)/AAA; HTL(252,253,254)/AAA; INV(255,256,257)/AAA; QSC(258,259,260)/AAA; GTK(261,262,263)/AAA; SQG(264,265,266)/AAA; ILN(267,268,269)/AAA; GFN(270,271,272)/AAA; KTV(273,274,275)/AAA; LRT(276,277,278)/AAA; LPR(279,280,281)/AAA; SKN(282,283,284)/AAA; F297A; GAA(308,309,310)/AAA; YRP(311,312,313)/AAA; TEE(314,315,316)/AAA; RSV(320,321,322)/AAA; GWG(323,324,325)/AAA; NIF(326,327,328)/AAA; FKH(332,333,334)/AAA; PE(313,315)/QA; RW(320,324)/AA; VRD(335,336,337)/AAA; P345A; F346A; F347A; Y349A; F356A; F361I; Y365A; GVC(366,367,368)/AAA; SMG(369,370,371)/AAA; LER(372,373,374)/AAA; Y377A; Y382A; PR(426,427)/AAA; F420A; F421A; W422A; PRV(426,427,428)/AAA; FYF(435,436,437)/AAA; WF(433,437)/AA; W442A; Y461A; EDK(462,463,464)/AAA; ERQ(465,466,467)/AAA; DFI(468,469,470)/AAA; FT(471,472)/AA; W476A; Y486A; MKK(493,494,496)/AAA; K494A; Y511A; EQK(515,516,517)/AAA; QQ(519,520)/RR; PP(524,527)/QQ; PRI(527,528,529)/AAA; PP(527,530)/QQ; Y539A; Y541A; GEQ(554,555,556)/AAA; GQG(557,558,559)/AAA; DC(560,561)/AA; EDE(563,564,565)/AAA; PQG(567,568,570)/AAA; PLG(571,572,573)/AAA; EPP(575,576,579)/AAA; GPC(578,579,580)/AAA; RKP(581,582,583)/AAA; CPY(584,585,586)/AAA; EQL(587,588,590)/AAA; GGD(591,592,593)/AAA; Y(8,40,52,53,94,99,158,159,190,191,193,196,541,586)/F; S(187,212,432,547,550)/A + T(93,160,314)/A; PP(5,6)/QQ + PP(6,9)/QQ + PPP(26,29,32)/QQQ + YN(40,42)/AA; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably PP(6,9)/QQ; Y8A; GPQ(15,16,17)/AAA; GDE(18,19,20)/AAA; GVP(24,25,26)/AAA; DGP(27,28,29)/AAA; VGY(37,38,40)/AAA; RR(50,51)/AA; RR(54,55)/AA; KRL(56,57,58)/AAA; Y75A; Y78A; QMQ(83,84,85)/AAA; LIL(86,87,88)/AAA; HYD(89,90,91)/AAA; ETY(92,93,94)/AAA; KYG(98,99,100)/AAA; NMG(101,102,103)/AAA; Y94A; RK(95,98)/AA; YN(99,101)/AA; Y125A; P127A; F132A; F133A; GTK(134,135,136)/AAA; WMM(137,138,139)/AAA; F140A; Y146A; F149A; W155A; E156A; YYT(158,159,160)/AAA; T160A; P163A; P174A; W176A; TRM(184,185,186)/AAA; YSH(193,194,195)/AAA; QGP(202,203,204)/AAA; S187A; Y190A; Y193A; PP(208,209)/QQ; RGS(210,211,212)/AAA; HPY(213,215,216)/AAA; F220A; F224A; Y225A; F227A; F228A; H229A; F232A; P238A; IYF(240,241,242)/AAA; YLY(246,247,248)/AAA; DLN(249,250,251)/AAA; HTL(252,253,254)/AAA; QSC(258,259,260)/AAA; GFN(270,271,272)/AAA; KTV(273,274,275)/AAA; LRT(276,277,278)/AAA; F297A; GAA(308,309,310)/AAA; YRP(311,312,313)/AAA; TEE(314,315,316)/AAA; RSV(320,321,322)/AAA; GWG(323,324,325)/AAA; NIF(326,327,328)/AAA; PE(313,315)/QA; RW(320,324)/AA; VRD(335,336,337)/AAA; P345A; F346A; F347A; Y349A; F356A; Y365A; GVC(366,367,368)/AAA; LER(372,373,374)/AAA; Y377A; Y382A; PR(426,427)/AAA; F420A; F421A; W422A; PRV(426,427,428)/AAA; W442A; Y461A; EDK(462,463,464)/AAA; ERQ(465,466,467)/AAA; DFI(468,469,470)/AAA; FT(471,472)/AA; W476A; Y486A; MKK(493,494,496)/AAA; K494A; Y511A; EQK(515,516,517)/AAA; CPY(584,585,586)/AAA; EQL(587,588,590)/AAA; Y(8,40,52,53,94,99,158,159,190,191,193,196,541,586)/F; S(187,212,432,547,550)/A + T(93,160,314)/A; PP(5,6)/QQ + PP(6,9)/QQ + PPP(26,29,32)/QQQ + YN(40,42)/AA; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

In some embodiments, the present invention is directed to a method of decreasing the trafficking of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); RW(320,324); VRD(335,336,337); Y365; GVC(366,367,368); LER(372,373,374); W442; ERQ(465,466,467); MKK(493,494,496); EQK(515,516,517); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the trafficking of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; LPR(279,280,281)/AAA; SKN(282,283,284)/AAA; FKH(332,333,334)/AAA; RW(320,324)/AA; VRD(335,336,337)/AAA; F361I; Y365A; GVC(366,367,368)/AAA; SMG(369,370,371)/AAA; LER(372,373,374)/AAA; W442A; ERQ(465,466,467)/AAA; MKK(493,494,496)/AAA; EQK(515,516,517)/AAA; PP(524,527)/QQ; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; RW(320,324)/AA; VRD(335,336,337)/AAA; Y365A; GVC(366,367,368)/AAA; LER(372,373,374)/AAA; W442A; ERQ(465,466,467)/AAA; MKK(493,494,496)/AAA; EQK(515,516,517)/AAA; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

In some embodiments, the present invention is directed to a method of decreasing the signaling and trafficking of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); RW(320,324); VRD(335,336,337); Y365; GVC(366,367,368); LER(372,373,374); W442; ERQ(465,466,467); MKK(493,494,496); EQK(515,516,517); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the signaling and trafficking of a given TLR7 in a cell or subject, which comprises administering to the cell or subject an Unc93b1 therapeutic, such as a mutant Unc93b1 protein, wherein its unmutated wildtype Unc93b1 protein sequence is natively associated with the given TLR7 and the mutant Unc93b1 protein comprises at least one amino acid mutation corresponding to one of the following mutations of SEQ ID NO: 1: Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; LPR(279,280,281)/AAA; SKN(282,283,284)/AAA; FKH(332,333,334)/AAA; RW(320,324)/AA; VRD(335,336,337)/AAA; F361I; Y365A; GVC(366,367,368)/AAA; SMG(369,370,371)/AAA; LER(372,373,374)/AAA; W442A; ERQ(465,466,467)/AAA; MKK(493,494,496)/AAA; EQK(515,516,517)/AAA; PP(524,527)/QQ; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; RW(320,324)/AA; VRD(335,336,337)/AAA; Y365A; GVC(366,367,368)/AAA; LER(372,373,374)/AAA; W442A; ERQ(465,466,467)/AAA; MKK(493,494,496)/AAA; EQK(515,516,517)/AAA; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

### Treating Aberrant TLR7 Activity with Unc93b1 Therapeutics

The Unc93b1^{PKP} mutation (i.e., PKP(530,531,532)/AAA) was introduced into the germline of mice using Cas9 genome editing methods in the art. This mutation disrupts interaction between Syntenin-1 and Unc93b1. An Unc93b1^{WT/PKP} founder was backcrossed to C57BL/6J for 1 generation, and then Unc93b1^{WT/PKP} mice were intercrossed to generate Unc93b1^{WT/WT}, Unc93b1^{WT/PKP}, and Unc93b1^{PKP/PKP} offspring for analysis. Unc93b1^{PKP/PKP} mice were born below the expected Mendelian frequency and were severely runted. The Unc93b1^{PKP/PKP} mice exhibited hallmarks of systemic inflammation and autoimmunity in TLR7 overexpressing mice, including increased frequencies of activated T cells, loss of marginal zone (MZ) B cells, increased frequencies of MHC^{hi} dendritic cells and inflammatory monocytes in secondary lymphoid organs, and development of emergency granulopoiesis within the bone marrow. Unc93b1^{PKP/PKP} mice developed anti-nuclear antibodies (ANA) very early in life. Unc93b1^{WT/PKP} mice also showed signs of immune dysregulation but not to the same extent as Unc93b1^{PKP/PKP} mice. Additionally, bone marrow-derived dendritic cells (BM-DCs), macrophages (BMMs), and B cells from Unc93b1^{WT/PKP} and Unc93b1^{PKP/PKP} mice mounted stronger responses to TLR7 ligands compared to Unc93b1^{WT/WT} cells, while responses to TLR9 and TLR4 ligands were about the same. Enhanced responses to R848 were most evident at low ligand concentrations. In line with the enhanced cytokine production, macrophages from Unc93b1^{PKP/PKP} mice showed stronger assembly of the Myddosome complex downstream of TLR7 activation. These enhanced TLR7 responses were not due to differences in Unc93b1 expression, as Unc93b1 protein levels were similar in BMMs from Unc93b1^{WT/WT}, Unc93b1^{WT/PKP} and Unc93b1^{PKP/PKP} mice.

These results demonstrate that Unc93b1 therapeutics impact the function of TLR7 in vivo without the need for an exogenous TLR7 ligand (e.g., a TLR7 agonist or antagonist). Thus, one or more Unc93b1 therapeutics that decrease or abolish TLR7 trafficking and/or signaling can be used to treat diseases and disorders caused by abnormally high TLR7 expression or activity. Conversely, one or more Unc93b1 therapeutics that increase TLR7 trafficking and/or signaling can be used to treat diseases and disorders caused by abnormally low TLR7 expression or activity. Methods in the art may be used to administer the one or more Unc93b1 therapeutics to a subject. For example, a subject may be administered a mutant Unc93b1 protein by way of administering a pharmaceutical composition comprising the mutant Unc93b1 protein, engrafting one or more cells, such as stem cells or T cells, that have been modified to express the mutant Unc93b1 protein, and/or manipulating the subject’s endogenous Unc93b1 gene such that it encodes the mutant Unc93b1 protein.

One skilled in the art may readily select one or more Unc93b1 therapeutics to be administered based on the desired therapeutic goal. For example, where the disease or disorder to be treated is the result of abnormally high TLR7 trafficking, one would select a mutant Unc93b1 protein (which its unmutated wildtype Unc93b1 protein sequence is natively associated with the given TLR7) that comprises at least one amino acid mutation corresponding to one of the following mutations of SEQ ID NO: 1: Where little to no trafficking is desired — QMQ(83,84,85); LIL(86,87,88); HYD(89,90,91); ETY(92,93,94); GTK(134,135,136); YLY(246,247,248); QSC(258,259,260); Y365; GVC(366,367,368); W442; Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); RW(320,324); or RR(50,51) + RRR(54,55,57) + RR(339,341); or Where about 25% of trafficking is desired - Y75; KYG(98,99,100); Y94; RK(95,98); IYF(240,241,242); LRT(276,277,278); LER(372,373,374); EQK(515,516,517); VRD(335,336,337); ERQ(465,466,467); or MKK(493,494,496).

As another example, where the disease or disorder to be treated is the result of abnormally high TLR7 signaling, one would select a mutant Unc93b1 protein (which its unmutated wildtype Unc93b1 protein sequence is natively associated with the given TLR7) that comprises at least one amino acid mutation corresponding to one of the following mutations of SEQ ID NO: 1: Where little to no signaling is desired — QMQ(83,84,85); LIL(86,87,88); HYD(89,90,91); ETY(92,93,94); GTK(134,135,136); WMM(137,138,139); E156; YLY(246,247,248); QSC(258,259,260); GFN(270,271,272); KTV(273,274,275); Y365; GVC(366,367,368); W442; Y486; Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); or PPP(524,527,530) + KPK(531,532,535) + Y541 + PP(576,579); Where about 25% of signaling is desired — KRL(56,57,58); Y75; KYG(98,99,100); Y94; RK(95,98); P127; F133; F140; F149; W155; T160; P174; W176; TRM(184,185,186); IYF(240,241,242); LRT(276,277,278); PE(313,315); RW(320,324); F356; LER(372,373,374); F420; EQK(515,516,517); or RR(50,51) + RRR(54,55,57) + RR(339,341); Where about 50% of signaling is desired — GDE(18,19,20); GVP(24,25,26); Y78; NMG(101,102,103); Y125; F132; Y146; YSH(193,194,195); Y190; PP(208,209); F224; Y225; F228; H229; F232; P238; DLN(249,250,251); HTL(252,253,254); YRP(311,312,313); GWG(323,324,325); VRD(335,336,337); P345; F347; Y349; Y382; PR(426,427); W422; PRV(426,427,428); EDK(462,463,464); DFI(468,469,470); FT(471,472); or Y511, or Where about 75% of signaling is desired — PP(6,9); Y8; GPQ(15,16,17); DGP(27,28,29); VGY(37,38,40); RR(50,51); RR(54,55); YN(99,101); YYT(158,159,160); P163; QGP(202,203,204); S187; Y193; RGS(210,211,212); HPY(213,215,216); F220; F227; F297; GAA(308,309,310); TEE(314,315,316); RSV(320,321,322); NIF(326,327,328); F346; Y377; F421; Y461; ERQ(465,466,467); W476; MKK(493,494,496); K494; CPY(584,585,586); EQL(587,588,590); or PP(5,6) + PP(6,9) + PPP(26,29,32) + YN(40,42).

As another example; where the disease or disorder to be treated is the result of abnormally low TLR7 signaling, one would select a mutant Unc93b1 protein (which its unmutated wildtype Unc93b1 protein sequence is natively associated with the given TLR7) that comprises at least one amino acid mutation corresponding to one of the following mutations of SEQ ID NO: 1: Where about a 125% increase in signaling is desired — DEL(34,35,36); R157; YKE(196,197,198); Y191; Y196; RR(339,341); F352; LQH(429,430,431); W477; K496; S(187,212,432,547,550); Where about a 150% increase in signaling is desired - EPL(30,32,33); IDL(317,318,319); LRH(340,341,342); P404; or SWI(432,433,434); where about a 175% increase in signaling is desired — QDE(199,200,201); or Where about a 200% or greater increase in signaling is desired — EEEEE(45,46,47,48,49)A; YY(52,53); REV(95,96,97); LPD(104,105,106); IDS(107,108,109); K110; or QLP(329,330,331).

### Modulating T Cell Activity With Unc93b1 Therapeutics

Using genetically-engineered mice to evaluate the impact of Unc93b1 mutations on various TLRs in vivo, it was surprisingly found that CD8⁺ T cells and CD4⁺ CD25⁺ T cells (Tregs) express TLR7 but not other TLRs 2, 4, 5, or 9. Preliminary studies indicate that TLR7 is functional on CD8⁺ and CD4⁺ T cells, including regulatory T cells (Tregs), and that TLR7 signaling modulates T cell function.

### 1. CD8⁺ and CD4⁺Foxp3⁻ T Cells

Preliminary in vitro studies demonstrate that CD8⁺ T cells proliferate and produce effector cytokines at a higher level when receiving both TCR (CD3) and TLR7 stimulation when compared to TCR stimulation alone. Specifically, CD8⁺ T cells were isolated from both wildtype and TLR7^{-/-} mice, labeled with different congenic markers, and stimulated together at a 1:1 ratio under the following conditions: 1) no stimulation, 2) TCR stimulation alone, 3) TCR stimulation plus CD28 (traditional CD8⁺ co-stimulation), and 4) TCR stimulation plus the TLR7 ligand, R848. Following three days in culture, similar numbers of wildtype and TLR7^{-/-} CD8⁺ T cells were present in the unstimulated, CD3 alone, and CD3/CD28 conditions. In contrast, when R848 was added to the culture, expansion of wildtype CD8⁺ T cells outpaced the TLR7^{-/-} CD8⁺ T cells by a significant margin. Wildtype CD8⁺ T cells also produced higher levels of the effector cytokines IFNγ and TNFα than their TLR7^{-/-} counterparts. Indeed, proliferation and cytokine production of CD8⁺ T cells receiving TCR and TLR7 stimuli was greater than that observed in the presence of TCR stimulation alone, and similar to levels observed upon TCR stimulation in combination with CD28. These results indicate that TLR7 signaling acts as an alternative costimulatory pathway for CD8⁺ T cells.

These results indicate that Unc93b1 mutations that increase the trafficking and/or signaling of TLR7 may be used to effect co-stimulation of CD8⁺ T cells. Therefore, one or more mutant Unc93b1 proteins may be administered to a subject to co-stimulate the subject’s CD8⁺ T cells.

### 2. Regulatory T Cells

Preliminary studies indicate that TLR7 signaling reduces the suppressive capacity of Tregs in vitro and possibly even in vivo. Specifically, in vitro suppression assays were used to evaluate the impact of TLR7 signaling on the ability of Tregs to suppress expansion of naive/effector T cells. The results indicate that TLR7 signaling reduces the suppression of T cells in a dose-dependent manner. This finding was confirmed in vivo using two distinct but related in vivo models of Treg function: 1) Treg suppression of homeostatic proliferation, and 2) Treg suppression of colitis. In both instances, CD4⁺ CD25⁻ CD4⁺Foxp3⁻ T cells are transferred into Rag1^{-/-} hosts (completely deficient in T and B cells). The CD4⁺Foxp3⁻ CD25⁻ T cells include effector T cells but lack Tregs, and will initially expand to “fill” the T cell compartment, a process termed homeostatic proliferation. If left unchecked for 6-8 weeks, the CD4⁺Foxp3⁻ T cells will differentiate into Th17 T cells and induce colitis. In both cases, the addition of Tregs will alter the outcome, either by reducing the total number of T cells present in homeostatic proliferation, or by inhibiting or reducing the severity of colitis. Preliminary experiments suggest that the results of the in vitro suppression assays are recapitulated in vivo. Specifically, Tregs lacking TLR7 suppressed colitis to an even greater extent than unaltered Tregs. Therefore, Unc93b1 mutations that inhibit or reduce TLR7 signaling and/or trafficking can be used to inhibit or treat colitis.

These results also indicate that Unc93b1 mutations that modulate the trafficking and/or signaling of TLR7 may be used to modulate the suppressive activity of Tregs. That is, Unc93b1 mutations that decrease or abolish TLR7 trafficking and/or signaling may be used to increase the suppressive activity of Tregs and Unc93b1 mutations that increase TLR7 trafficking and/or signaling may be used to decrease the suppressive activity of Tregs.

Additionally, in vitro stimulation of Tregs with TLR7 ligand causes proliferation, increased expression of the IL-18 and IL-33 receptors, and production of amphiregulin— hallmarks of Tregs that facilitate repair of damaged tissue following injury or infection, e.g., lung repair after influenza infection. In fact, preliminary experiments showed that during influenza infection, mice with a Treg-specific deletion of TLR7 exhibit a significantly elevated level of inflammatory cell recruitment to the lungs, as well as exaggerated weight loss and reduced blood oxygen saturation compared to controls, i.e., mice having wildtype TLR7. These results indicate that TLR7 signaling in Tregs primes them to perform tissue repair. Thus, Unc93b1 mutations that increase TLR7 trafficking and/or signaling can be used to increase, enhance, or induce tissue repair by Tregs. Unc93b 1 mutations that increase TLR7 trafficking and/or signaling can be used induce or increase Treg proliferation, increased expression of IL-18 and IL-33 receptors, and induce or increase of amphiregulin production.

Therefore, Unc93b1 mutations may be used to modulate the activity of CD8⁺ T cells, CD4⁺ T cells, Tregs, and/or CAR T cells/Tregs. For example, Unc93b1 mutations that increase TLR7 trafficking and/or signaling may be used to provide co-stimulation by way of TLR7 signaling in CD8⁺ T cells and/or CAR T cells or increase or induce their tissue repair functions. Similarly, Unc93b1 mutations that increase TLR7 trafficking and/or signaling may be used to promote cytokine production by enhancing TLR7 signaling in dendritic cells. Such methods may be used to enhance a subject’s overall immunity to viruses, bacteria, and other pathogens or used as an adjuvant to enhance the efficacy of immunotherapeutics, e.g., cancer immunotherapeutics and vaccines.

Sustained TLR7 signaling in Tregs induces cell expansion and expression of genes important for tissue repair. Therefore, Tregs may be “re-programmed” by being modified to have one or more Unc93b 1 mutations to result in sustained TLR7 signaling and then used to treat tissue damage and/or promote tissue repair.

### TLR8

TLR7 and TLR8 are the most closely related of the toll-like receptors. TLR7 and TLR8 are closely related phylogentically, representing a tandem duplication in the genomes of humans, mice, and other vertebrates. TLR7 and TLR8 recognize single-stranded RNA in endosomes, which is a common feature of viruses such as HIV, influenza, and HCV. TLR7 and TLR8 also occupy the same genetic zipcode as next-door neighbors on the X-chromosome. Numerous ligands that stimulate both TLR7 and TLR8 are known and include viral ssRNAs, imidazoquinoline compounds, and guanosine analogs. In addition, overexpression of either mouse TLR7 or human TLR8 in mice leads to autoimmune inflammation. Taken together, the nearly identical functionality, ligand specificity, genomic location, and evolutionary relationship of TLR7 and TLR8 strongly suggest a shared regulatory mechanism.

Therefore, Unc93b 1 mutations that modulate the trafficking and signaling of TLR7 are expected to similarly modulate the trafficking and signaling of TLR8. As such, in some embodiments, the present invention is directed to a method of increasing the signaling of a given TLR8 in a cell or subject, which comprises administering to the cell or subject an Unc93b1 therapeutic, such as a mutant Unc93b1 protein, wherein its unmutated wildtype Unc93b 1 protein sequence is natively associated with the given TLR8 and the mutant Unc93b1 protein comprises at least one amino acid mutation corresponding to one of the following mutations of SEQ ID NO: 1: EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; KPK(531,532,535); QHK(533,534,535); VRG(536,537,538); LEE(542,543,544); DME(551,552,553); and/or S(187,212,432,547,550), preferably EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; and/or S(187,212,432,547,550), more preferably EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; and/or S(187,212,432,547,550).

In some embodiments, the present invention is directed to a method of increasing the signaling of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: EPL(30,32,33)/AAA; DEL(34,35,36)/AAA; EEEEE(45,46,47,48,49)/AAAAA; YY(52,53)/AA; REV(95,96,97)/AAA; LPD(104,105,106)/AAA; IDS(107,108,109)/AAA; K110A; R157A; YKE(196,197,198)/AAA; QDE(199,200,201)/AAA; Y191A; Y196A; IDL(317,318,319)/AAA; QLP(329,330,331)/AAA; RR(339,341)/AA; LRH(340,341,342)/AAA; F352A; P404A; LQH(429,430,431)/AAA; SWI(432,433,434)/AAA; W477A; K496A; W513R; KPK(531,532,535)/AAA; QHK(533,534,535)/AAA; VRG(536,537,538)/AAA; LEE(542,543,544)/AAA; DME(551,552,553)/AAA; and/or S(187,212,432,547,550)/A, preferably EPL(30,32,33)/AAA; DEL(34,35,36)/AAA; EEEEE(45,46,47,48,49)/AAAAA; YY(52,53)/AA; REV(95,96,97)/AAA; LPD(104,105,106)/AAA; IDS(107,108,109)/AAA; K110A; R157A; YKE(196,197,198)/AAA; QDE(199,200,201)/AAA; Y191A; Y196A; IDL(317,318,319)/AAA; QLP(329,330,331)/AAA; RR(339,341)/AA; LRH(340,341,342)/AAA; F352A; P404A; LQH(429,430,431)/AAA; SWI(432,433,434)/AAA; W477A; K496A; and/or S(187,212,432,547,550)/A.

In some embodiments, the present invention is directed to a method of decreasing the signaling of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: EVE(2,3,4); PP(6,9); Y8; VG(10, 12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); PP(524,527); PRI(527,528,529); PP(527,530); Y539; Y541; GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably EVE(2,3,4); PP(6,9); Y8; VG(10, 12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), more preferably PP(6,9); Y8; GPQ(15,16,17); GDE(18,19,20); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); YLY(246,247,248); DLN(249,250,251); HTL(252,253,254); QSC(258,259,260); GFN(270,271,272); KTV(273,274,275); LRT(276,277,278); F297; GAA(308,309,310); YRP(311,312,313); TEE(314,315,316); RSV(320,321,322); GWG(323,324,325); NIF(326,327,328); PE(313,315); RW(320,324); VRD(335,336,337); P345; F346; F347; Y349; F356; Y365; GVC(366,367,368); LER(372,373,374); Y377; Y382; PR(426,427); F420; F421; W422; PRV(426,427,428); W442; Y461; EDK(462,463,464); ERQ(465,466,467); DFI(468,469,470); FT(471,472); W476; Y486; MKK(493,494,496); K494; Y511; EQK(515,516,517); CPY(584,585,586); EQL(587,588,590); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the signaling of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: EVE(2,3,4)/AAA; PP(6,9)/QQ; Y8A; VG(10,12)/AA; GPQ(15,16,17)/AAA; GDE(18,19,20)/AAA; DRH(21,22,23)/AAA; GVP(24,25,26)/AAA; DGP(27,28,29)/AAA; VGY(37,38,40)/AAA; RR(50,51)/AA; RR(54,55)/AA; KRL(56,57,58)/AAA; Y75A; Y78A; QMQ(83,84,85)/AAA; LIL(86,87,88)/AAA; HYD(89,90,91)/AAA; ETY(92,93,94)/AAA; KYG(98,99,100)/AAA; NMG(101,102,103)/AAA; Y94A; RK(95,98)/AA; YN(99,101)/AA; Y125A; P127A; F132A; F133A; GTK(134,135,136)/AAA; WMM(137,138,139)/AAA; F140A; Y146A; F149A; W155A; E156A; YYT(158,159,160)/AAA; T160A; P163A; P174A; W176A; TRM(184,185,186)/AAA; SQK(187,188,189)/AAA; YYE(190,191,192)/AAA; YSH(193,194,195)/AAA; QGP(202,203,204)/AAA; S187A; Y190A; Y193A; PP(208,209)/QQ; RGS(210,211,212)/AAA; HPY(213,215,216)/AAA; F220A; F224A; Y225A; F227A; F228A; H229A; F232A; P238A; IYF(240,241,242)/AAA; LNN(243,244,245)/AAA; YLY(246,247,248)/AAA; DLN(249,250,251)/AAA; HTL(252,253,254)/AAA; INV(255,256,257)/AAA; QSC(258,259,260)/AAA; GTK(261,262,263)/AAA; SQG(264,265,266)/AAA; ILN(267,268,269)/AAA; GFN(270,271,272)/AAA; KTV(273,274,275)/AAA; LRT(276,277,278)/AAA; LPR(279,280,281)/AAA; SKN(282,283,284)/AAA; F297A; GAA(308,309,310)/AAA; YRP(311,312,313)/AAA; TEE(314,315,316)/AAA; RSV(320,321,322)/AAA; GWG(323,324,325)/AAA; NIF(326,327,328)/AAA; FKH(332,333,334)/AAA; PE(313,315)/QA; RW(320,324)/AA; VRD(335,336,337)/AAA; P345A; F346A; F347A; Y349A; F356A; F361I; Y365A; GVC(366,367,368)/AAA; SMG(369,370,371)/AAA; LER(372,373,374)/AAA; Y377A; Y382A; PR(426,427)/AAA; F420A; F421A; W422A; PRV(426,427,428)/AAA; FYF(435,436,437)/AAA; WF(433,437)/AA; W442A; Y461A; EDK(462,463,464)/AAA; ERQ(465,466,467)/AAA; DFI(468,469,470)/AAA; FT(471,472)/AA; W476A; Y486A; MKK(493,494,496)/AAA; K494A; Y511A; EQK(515,516,517)/AAA; QQ(519,520)/RR; PP(524,527)/QQ; PRI(527,528,529)/AAA; PP(527,530)/QQ; Y539A; Y541A; GEQ(554,555,556)/AAA; GQG(557,558,559)/AAA; DC(560,561)/AA; EDE(563,564,565)/AAA; PQG(567,568,570)/AAA; PLG(571,572,573)/AAA; EPP(575,576,579)/AAA; GPC(578,579,580)/AAA; RKP(581,582,583)/AAA; CPY(584,585,586)/AAA; EQL(587,588,590)/AAA; GGD(591,592,593)/AAA; Y(8,40,52,53,94,99,158,159,190,191,193,196,541,586)/F; S(187,212,432,547,550)/A + T(93,160,314)/A; PP(5,6)/QQ + PP(6,9)/QQ + PPP(26,29,32)/QQQ + YN(40,42)/AA; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably PP(6,9)/QQ; Y8A; GPQ(15,16,17)/AAA; GDE(18,19,20)/AAA; GVP(24,25,26)/AAA; DGP(27,28,29)/AAA; VGY(37,38,40)/AAA; RR(50,51)/AA; RR(54,55)/AA; KRL(56,57,58)/AAA; Y75A; Y78A; QMQ(83,84,85)/AAA; LIL(86,87,88)/AAA; HYD(89,90,91)/AAA; ETY(92,93,94)/AAA; KYG(98,99,100)/AAA; NMG(101,102,103)/AAA; Y94A; RK(95,98)/AA; YN(99,101)/AA; Y125A; P127A; F132A; F133A; GTK(134,135,136)/AAA; WMM(137,138,139)/AAA; F140A; Y146A; F149A; W155A; E156A; YYT(158,159,160)/AAA; T160A; P163A; P174A; W176A; TRM(184,185,186)/AAA; YSH(193,194,195)/AAA; QGP(202,203,204)/AAA; S187A; Y190A; Y193A; PP(208,209)/QQ; RGS(210,211,212)/AAA; HPY(213,215,216)/AAA; F220A; F224A; Y225A; F227A; F228A; H229A; F232A; P238A; IYF(240,241,242)/AAA; YLY(246,247,248)/AAA; DLN(249,250,251)/AAA; HTL(252,253,254)/AAA; QSC(258,259,260)/AAA; GFN(270,271,272)/AAA; KTV(273,274,275)/AAA; LRT(276,277,278)/AAA; F297A; GAA(308,309,310)/AAA; YRP(311,312,313)/AAA; TEE(314,315,316)/AAA; RSV(320,321,322)/AAA; GWG(323,324,325)/AAA; NIF(326,327,328)/AAA; PE(313,315)/QA; RW(320,324)/AA; VRD(335,336,337)/AAA; P345A; F346A; F347A; Y349A; F356A; Y365A; GVC(366,367,368)/AAA; LER(372,373,374)/AAA; Y377A; Y382A; PR(426,427)/AAA; F420A; F421A; W422A; PRV(426,427,428)/AAA; W442A; Y461A; EDK(462,463,464)/AAA; ERQ(465,466,467)/AAA; DFI(468,469,470)/AAA; FT(471,472)/AA; W476A; Y486A; MKK(493,494,496)/AAA; K494A; Y511A; EQK(515,516,517)/AAA; CPY(584,585,586)/AAA; EQL(587,588,590)/AAA; Y(8,40,52,53,94,99,158,159,190,191,193,196,541,586)/F; S(187,212,432,547,550)/A + T(93,160,314)/A; PP(5,6)/QQ + PP(6,9)/QQ + PPP(26,29,32)/QQQ + YN(40,42)/AA; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

In some embodiments, the present invention is directed to a method of decreasing the trafficking of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); RW(320,324); VRD(335,336,337); Y365; GVC(366,367,368); LER(372,373,374); W442; ERQ(465,466,467); MKK(493,494,496); EQK(515,516,517); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the trafficking of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; LPR(279,280,281)/AAA; SKN(282,283,284)/AAA; FKH(332,333,334)/AAA; RW(320,324)/AA; VRD(335,336,337)/AAA; F361I; Y365A; GVC(366,367,368)/AAA; SMG(369,370,371)/AAA; LER(372,373,374)/AAA; W442A; ERQ(465,466,467)/AAA; MKK(493,494,496)/AAA; EQK(515,516,517)/AAA; PP(524,527)/QQ; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; RW(320,324)/AA; VRD(335,336,337)/AAA; Y365A; GVC(366,367,368)/AAA; LER(372,373,374)/AAA; W442A; ERQ(465,466,467)/AAA; MKK(493,494,496)/AAA; EQK(515,516,517)/AAA; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

In some embodiments, the present invention is directed to a method of decreasing the signaling and trafficking of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341), preferably Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); RW(320,324); VRD(335,336,337); Y365; GVC(366,367,368); LER(372,373,374); W442; ERQ(465,466,467); MKK(493,494,496); EQK(515,516,517); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and/or RR(50,51) + RRR(54,55,57) + RR(339,341).

In some embodiments, the present invention is directed to a method of decreasing the signaling and trafficking of a given TLR8 in a cell or subject, which comprises administering to the cell or subject an Unc93b1 therapeutic, such as a mutant Unc93b1 protein, wherein its unmutated wildtype Unc93b1 protein sequence is natively associated with the given TLR8 and the mutant Unc93b1 protein comprises at least one amino acid mutation corresponding to one of the following mutations of SEQ ID NO: 1: Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; LPR(279,280,281)/AAA; SKN(282,283,284)/AAA; FKH(332,333,334)/AAA; RW(320,324)/AA; VRD(335,336,337)/AAA; F361I; Y365A; GVC(366,367,368)/AAA; SMG(369,370,371)/AAA; LER(372,373,374)/AAA; W442A; ERQ(465,466,467)/AAA; MKK(493,494,496)/AAA; EQK(515,516,517)/AAA; PP(524,527)/QQ; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA, preferably Y75A; QMQ(83,84,85)/AAA; LIL(86,87,88)/AAA; HYD(89,90,91)/AAA; ETY(92,93,94)/AAA; KYG(98,99,100)/AAA; Y94A; RK(95,98)/AA; GTK(134,135,136)/AAA; IYF(240,241,242)/AAA; YLY(246,247,248)/AAA; QSC(258,259,260)/AAA; LRT(276,277,278)/AAA; RW(320,324)/AA; VRD(335,336,337)/AAA; Y365A; GVC(366,367,368)/AAA; LER(372,373,374)/AAA; W442A; ERQ(465,466,467)/AAA; MKK(493,494,496)/AAA; EQK(515,516,517)/AAA; Y(8,40,52,53,94,99,158,159,190,191,193,196,541,586)/F; S(187,212,432,547,550)/A + T(93,160,314)/A; TY(93,94)/AA + REK(95,96,98)/AAA + YN(99,101)/AA; YF(241,242)/AA + YL(246,247)/AA; PE(313,315)/QA + RW(320,324)/AA + FF(328,332)/AA; PPP(524,527,530)/QQQ + KPK(531,532,535)/AAA + Y541A + PP(576,579)/QQ; and/or RR(50,51)/AA + RRR(54,55,57)/AAA + RR(339,341)/AA.

### Additional Therapeutic Applications of Unc93b1 Therapeutics

Increased TLR7 expression and/or increased stimulation of TLR7 promotes tumor control and suppresses or inhibits metastases, enhances memory B cell expansion, boosts dendritic cell responses, improves the efficacy vaccines (e.g., pertussis vaccine), facilitates immune responses against a variety of different pathogens (e.g., HIV, West Nile Virus, Influenzavirus, Japanese Encephalitis Virus, Ross River Virus, Plasmodium spp., Hepatitis viruses, and Arenaviruses), suppresses inflammatory responses, and suppresses asthma symptoms. Therefore, in some embodiments, one or more Unc93b1 therapeutics that increase the trafficking and/or signaling of TLR7 may be administered to a subject to promote tumor control, suppress or inhibit metastases, enhance memory B cell expansion, boost dendritic cell responses, improve the efficacy of a vaccine, facilitate an immune response against a pathogen such as HIV, West Nile Virus, an Influenzavirus, Japanese Encephalitis Virus, Ross River Virus, Plasmodium spp., a Hepatitis virus, or an Arenavirus), suppress an inflammatory response, and/or suppress asthma symptoms in the subject.

Reducing TLR7 signaling reduces or inhibits bone destruction in rheumatoid arthritis, may treat anemias and autoimmune disorders such as systemic lupus erythematosus, treats or inhibits plaque psoriasis, and increases CNS autoimmunity. Therefore, in some embodiments, one or more Unc93b1 therapeutics that decrease or abolish the trafficking and/or signaling of TLR7 may be administered to a subject to inhibit bone destruction from rheumatoid arthritis, treat anemias, treat autoimmune disorders such as systemic lupus erythematosus, treat or inhibit plaque psoriasis, and increase CNS autoimmunity in a subject.

As TLR8 expression and signaling can lead to autoimmune inflammation and because Unc93b1 mutations that modulate the trafficking and signaling of TLR7 are expected to similarly modulate the trafficking and signaling of TLR8, one or more Unc93b1 therapeutics that decrease or abolish the trafficking and/or signaling of TLR7 may be administered to a subject to treat or inhibit autoimmune inflammation resulting from TLR8 activity, e.g., RNA recognition by TLR8.

Because both TLR7 and TLR8 are involved in modulating a subject’s responsiveness to antivirals such as R-848 and because Unc93b1 mutations that modulate the trafficking and signaling of TLR7 are expected to similarly modulate the trafficking and signaling of TLR8, one or more Unc93b1 therapeutics may be administered to a subject to modulate the subject’s responsiveness to antiviral therapy.

### Mutant UNC93b1 Proteins

In some embodiments, the present invention is directed to mutant Unc93b1 proteins. As used herein, a “mutant Unc93b1 protein” refers to an Unc93b1 protein that has at least one amino acid mutation compared to its unmutated wildtype sequence. In some embodiments, preferred mutant Unc93b 1 proteins include those having an unmutated wildtype sequence comprising at least 90% sequence identity to SEQ ID NO: 1 (Accession Number Q8VCW4.2) or SEQ ID NO: 2 (Accession Number NP_112192.2) and at least one amino acid mutation that corresponds to one of the mutations provided in FIG. 1 when optimally aligned with SEQ ID NO: 1 (Accession Number Q8VCW4.2). In some embodiments, the amino acid sequence of the mutant Unc93b1 protein comprises at least 85%, at least 86%, at least 87%, at least 88%, or at least 89% sequence identity to SEQ ID NO: 1 (Accession Number Q8VCW4.2) or SEQ ID NO: 2 (Accession Number NP_112192.2). In some embodiments, the amino acid sequence of the mutant Unc93b1 protein comprises at least 95%, at least 96%, at least 97%, at least 98%, or at least 99% sequence identity to its unmutated wildtype sequence. In some embodiments, the amino acid sequence of the mutant Unc93b1 protein comprises less than 100% sequence identity to naturally occurring unc-93 homolog B1 proteins. It should, however, be noted that a mutant Unc93b1 protein (of a Unc93b1 therapeutic) administered according to the methods described herein may have 100% sequence identity to a naturally occurring unc-93 homolog B1 protein so long as the naturally occurring unc-93 homolog B1 protein is not natively associated with the TLR in the cell or subject to which the mutant Unc93b1 protein is intended to modulate. That is, for example, where the trafficking or signaling of human TLR7 is to be increased or decreased, the amino acid sequence of the mutant Unc93b1 protein being administered may be 100% identical to a naturally occurring chimpanzee unc-93 homolog B1 protein.

As provided herein, amino acid mutations are indicated by the amino acid residue (or residues) and their amino acid position based on the parental polypeptide (i.e., the wildtype or unmutated polypeptide) followed by the specific mutation. For example, as shown in FIG. 1, “Y365I” indicates that tyrosine residue at position 365 of a given reference sequence, e.g., Q8VCW4.2, is substituted with isoleucine. Thus, a “Y365” mutation indicates the amino acid residue of a given Unc93b1 protein that aligns with the tyrosine residue at position 365 of Q8VCW4.2, when the given Unc93b1 protein and Q8VCW4.2 are optimally aligned, is mutated. As another example, a “EQK(515,516,517)/AAA” mutation indicates that the amino acid residues of a given Unc93b1 protein that align with glutamic acid, glutamine, and lysine at amino acid positions 515, 516, and 517 of Q8VCW4.2, when the given Unc93b1 protein and Q8VCW4.2 are optimally aligned, are each substituted with alanine. An “EQK(515,516,517)” mutation indicates that the amino acid residues of a given Unc93b1 protein that aligns with glutamic acid, glutamine, and lysine at positions 515, 516, and 517 of Q8VCW4.2, when the given Unc93b1 protein and Q8VCW4.2 are optimally aligned, are each independently mutated. Similarly, a “T(93,160,314)/A” mutation indicates that the amino acid residues of a given Unc93b1 protein that align with the threonine residues at positions 93, 160, and 314 of Q8VCW4.2, when the given Unc93b1 protein and Q8VCW4.2 are optimally aligned, are each substituted with alanine. Thus, “T(93,160,314)” mutation indicates that the amino acid residues of a given Unc93b1 protein that align with the threonine residues at positions 93, 160, and 314 of Q8VCW4.2, when the given Unc93b1 protein and Q8VCW4.2 are optimally aligned, are each independently mutated. Amino acid mutations include substitutions, deletions, additions, and post-translational modifications (e.g., chemical modifications). In some embodiments, the amino acid mutations are preferably amino acid substitutions.

Mutant Unc93b1 proteins may be made using methods known in the art including chemical synthesis, biosynthesis or in vitro synthesis using recombinant DNA methods, and solid phase synthesis. See, e.g., Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. Mutant Unc93b1 proteins may be purified using protein purification techniques known in the art such as reverse phase high-performance liquid chromatography (HPLC), ion-exchange or immunoaffinity chromatography, filtration or size exclusion, or electrophoresis. See, e.g., Olsnes and Pihl (1973) Biochem. 12(16):3121-3126; and Scopes (1982) Protein Purification, Springer-Verlag, NY, which are herein incorporated by reference. Alternatively, the polypeptides may be made by recombinant DNA techniques known in the art. Thus, polynucleotides that encode mutant Unc93b1 proteins are contemplated herein. In some embodiments, the polypeptides and polynucleotides are isolated.

As used herein, an “isolated” compound refers to a compound that is isolated from its native environment. For example, an isolated polynucleotide is a one which does not have the bases normally flanking the 5′ end and/or the 3′ end of the polynucleotide as it is found in nature. As another example, an isolated protein fragment is a one which does not have its native amino acids, which correspond to the full-length polypeptide, flanking the N-terminus, C-terminus, or both.

### Kits

In some embodiments, the present invention provides kits comprising one or more Unc93b1 therapeutics, optionally in a composition or in combination with one or more supplementary agents, packaged together with one or more reagents or drug delivery devices for treating a subject in need thereof. In some embodiments, the kits comprise the one or more Unc93b1 therapeutics, optionally in one or more unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Compositions

Compositions, including pharmaceutical compositions, comprising one or more Unc93b1 therapeutics are contemplated herein. A composition generally comprises an effective amount of an active agent and a diluent and/or carrier. The term “pharmaceutical composition” refers to a composition suitable for pharmaceutical use in a subject. A pharmaceutical composition generally comprises a therapeutically effective amount of an active agent, e.g., one or more Unc93b 1 therapeutics as contemplated herein, and a pharmaceutically acceptable carrier. In addition to the one or more Unc93b 1 therapeutics, pharmaceutical compositions may include one or more supplementary agents. Examples of suitable supplementary agents include TLR7 ligands, TLR7 agonists, TLR7 antagonists, and the like.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount of a given therapeutic that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, the level of TLR7 trafficking and/or signaling in the subject, previous treatments, the general health and age of the subject, and the like. Nevertheless, effective amounts and therapeutically effective amounts may be readily determined by methods in the art.

The one or more Unc93b 1 therapeutics may be administered, preferably in the form of pharmaceutical compositions, to a subject. Preferably the subject is mammalian, more preferably, the subject is human. Preferred pharmaceutical compositions are those comprising at least one Unc93b 1 therapeutic in a therapeutically effective amount and a pharmaceutically acceptable vehicle. In some embodiments, a therapeutically effective amount of a mutant Unc93b1 protein ranges from about 0.01 to about 10 mg/kg body weight, about 0.01 to about 3 mg/kg body weight, about 0.01 to about 2 mg/kg, about 0.01 to about 1 mg/kg, or about 0.01 to about 0.5 mg/kg body weight for parenteral formulations. Therapeutically effective amounts for oral administration may be up to about 10-fold higher. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular Unc93b1 therapeutic used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the one or more Unc93b1 therapeutic calculated to produce the desired therapeutic effect in association with the required pharmaceutically acceptable carrier. The specification for the dosage unit forms of the invention are dictated by and directly dependent on the unique characteristics of the given Unc93b1 therapeutic and desired therapeutic effect to be achieved, and the limitations inherent in the art of compounding such an active compound for the treatment of individuals.

Toxicity and therapeutic efficacy of Unc93b1 therapeutics according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Unc93b1 therapeutics which exhibit large therapeutic indices are preferred. While Unc93b1 therapeutics that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more Unc93b1 therapeutics can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

### Exemplary Therapeutic Methods

In some embodiments, gene therapy methods in the art may be used to genetically modify the Unc93b1 gene in a subject to have one or more mutations as disclosed herein. See, e.g., Hultquist, et al. Alternatively, or in addition to, expression and signaling of a TLR of interest may be increased or decreased using gene therapy methods in the art, e.g., CRISPR editing, to genetically modify the gene encoding the TLR of interest. For example, if using CRISPR, TLR7-201 (ensembl.org) may be targeted to knockout human TLR7 and both TLR8-201 and TLR8-202 (ensembl.org) may be targeted to knockout human TLR8 using one or more suitable RNA guide sequences.

In some embodiments, the Unc93b1 gene in stem cells or T cells may be recombinantly modified to have one or more mutations as disclosed herein and then engrafted in a subject using methods in the art. See, e.g., Morgan & Boyerinas. Alternatively, or in addition to, recombinant methods in the art may be used to modify the TLR of interest in the stem cells or T cells, which are to be in engrafted, to exhibit the desired TLR expression and/or signaling.

In some embodiments, a therapeutically effective amount of one or more mutant Unc93b1 proteins or composition thereof may be administered to a subject. The administration may be local or systemic and by any suitable route, e.g., oral, injection, etc.

The following examples are intended to illustrate but not to limit the invention.

### METHODS

### Unc93b1 and TLR7

The accession number for the amino acid sequence of mouse Unc93b1 is Q8VCW4.2 (SEQ ID NO:1) and the accession number the amino acid sequence of human Unc93b1 is NP_112192.2 (SEQ ID NO: 2), both of which are herein incorporated by reference in their entirety. The accession numbers for the amino acid sequence of human TLR7 is NP_057646.1 and the amino acid sequence of mouse TLR7 is XP_006528776.1, both of which are herein incorporated by reference. The reference sequence of the mutations and amino acid locations exemplified herein is the mouse Unc93b1 sequence (Accession No. Q8VCW4.2, SEQ ID NO:1).

### Antibodies and Reagents

The following antibodies were used for immunoblots and immunoprecipitations: anti-HA as purified antibody or matrix (3F10, Roche), anti-FLAG as purified antibody or matrix (M2, Sigma-Aldrich), anti-mLamp-1 (AF4320, R&D Systems), anti-Calnexin (ADI-SPA-860, Enzo Life Sciences), anti-Gapdh (GT239, GeneTex), anti-Myd88 (AF3109, R&D Systems), anti-IRAK2 (Cell Signaling), anti-Phospho-p38 (Cell Signaling), anti-p38 (Cell Signaling), anti-Phospho-SAPK/JNK (81E11, Cell Signaling), anti-SAPK/JNK (56G8, Cell Siganling), anti-Phospho-p44/42 (ERK½) (D13.14.4E, Cell Signaling), anti-p44/42 (ERK½) (137F5, Cell Siganling), anti-IκBα (Cell Signaling), anti-Syntenin-1 (2C12, Novusbio), anti-Unc93b1 (PA5-20510, Thermo Scientific), anti-ubiquitin (P4D1, Santa Cruz), anti-K63-linked ubiquitin (human polyclonal, kind gift from Michael Rape), goat anti-mouse IgG-AlexaFluor680 (Invitrogen), goat anti-mouse IgG-AlexaFluor680 (Invitrogen), rabbit anti-goat IgG-AlexaFluor680 (Invitrogen), ygoat anti-human IRDye 680RD (Licor), goat anti-mouse IRDye 800CW (Licor), donkey anti-rabbit IRDye 680RD (Licor), goat anti-rat IRDye 800CW (Licor). Antibodies for immunofluorescence were: rat anti-HA (3F10, Roche), rabbit anti-Lamp1 (ab24170, Abcam), goat anti-rat IgG-AlexaFluor488 (Jackson Immunoresearch), goat anti-rabbit IgG-AlexaFluor647 (Jackson Immunoresearch). Cells were mounted in Vectashield Hard Set Mounting Medium for Fluorescence (Vector Laboratories). For ELISA: anti-mouse TNFα purified (1F3F3D4, eBioscience), anti-mouse TNFα-biotin (XT3/XT22, eBioscience), Streptavidin-HRP (BD Pharmingen). Antibodies and reagents used for flow cytometry were: anti-TNFα (MP6-XT22, eBioscience), purified anti-CD16/32 Fc Block (2.4G2), CD3ε (145-2C11, BioLegend), CD4 (GK1.5, BioLegend), CD8 (53-6.7, BioLegend), CD44 (IM7, eBioscience), CD62L (MEL-14, eBioscience), CD69 (H1.2F3, eBioscience), CD1d (1B1, eBioscience), B220 (RA3-6B2, Invitrogen), CD19 (6D5, BioLegend), IgD (11-26c.2a, BioLegend), IgM (eB121-15F9, eBioscience), CD21 (eBio8D9, eBioscience), CD23 (B3B4, eBioscience), CD138 (281-2, BioLegend), CD11b (M1/70, BioLegend), Ly6G (1A8, TONBO biosciences), Ly6C (HK1.4, BioLegend), F4/80 (CI:A3-1, AbD serotec), MHCII (M5/114.15.2, eBioscience), CD86 (GL1, eBioscience), CD11c (N418, BioLegend), CD117 (c-Kit) (2B8, eBioscience), Sca-1 (D7, eBioscience). For ANA detection: anti-mouse IgG-AlexaFluor 488 (Jackson Immunoresearch), anti-mouse IgM-FITC (Invitrogen).

The antibody against phosphorylated Unc93b1 was generated by Invitrogen against synthesized phospho-peptide (YLEEDN(pS)DE(pS)DMEGEQ (SEQ ID NO: 7)) using their “Rabbit, 90-Day immunization” protocol. Antibody in sera was enriched with immobilized phospho-peptide, followed by negative absorption with unphosphorylated peptide.

CpG-B (ODN1668: TCCATGACGTTCCTGATGCT (SEQ ID NO: 8), all phosphorothioate linkages) was synthesized by Integrated DNA Technologies. R848, PolyIC HMW, ssRNA40/LyoVec, and LPS were purchased from InvivoGen. Human IL-1b was from Invitrogen. NP-40 (Igepal CA-630) was from Sigma-Aldrich. Lipofectamine-LTX reagent (Invitrogen) and OptiMEM-I (Invitrogen) were used for transfection of plasmid DNA. ProMag 1 Series-COOH Surfactant free magnetic beads (#25029) for phagosome preparations were purchased from Polysciences. For luciferase assays: Renilla substrate: Coelenterazine native (Biotum), Firefly substrate: Luciferin (Biosynth), Passive Lysis Buffer, 5x (Promega).

### Animals

Mice were housed under specific-pathogen-free conditions at the University of California, Berkeley. All mouse experiments were performed in accordance with the guidelines of the Animal Care and Use Committee at UC Berkeley. Unless noted mice were analyzed at 5-8 weeks of age. C57BL/6J and TLR7^{-/-} mice (on the C57BL/6J background) were from the Jackson Laboratory. Unc93b1^{PKP} mice were generated using Cas9 genome editing. The guide RNA used was: TGCTGTGGCTTCGGAATGCGCGG (SEQ ID NO: 9). The single stranded oligo template contained 60 bp homology arms on both sides and four phosphothioate linkages at the ends (one at the 5′ and three at the 3′ end of the oligo). Briefly, female C57BL/6J mice at 4 weeks of age were superovulated and mated overnight with C57BL/6J male mice (>8 weeks old). Zygotes were harvested from superovulated females and were placed in KSOM medium (Millipore) before use. CRISPR/Cas9 mixture was prepared in final concentration of cas9 mRNA (100 ng/µl), sgRNA (50 ng/µl) and single stranded oligo (100 ng/µl). The CRISPR/Cas9 mixture was microinjected into 80 zygotes using a micromanipulator (Narishige) and microscope (Nikon). After microinjection, 67 embryos were transferred to three CD1 recipients via oviduct transfer. Offspring was genotyped by sequencing for the correct targeted allele and further bred to ensure germline transmission.

### Unc93b1 Library Design and Plasmid Constructs

The Unc93b1 mutagenesis library has been generated by Invitrogen. Briefly, the mouse Unc93b1 gene was optimized for the codon bias of Mus musculus and regions of very high (>80%) and very low (<30%) GC content have been avoided. The codon-optimized mouse Unc93b1 gene was c-terminally tagged with 3xFLAG (DYKDHDGDYKDHDIDYKDDDDK (SEQ ID NO: 10)) and subjected to a triple-alanine scanning mutagenesis spanning sequences corresponding to tail and loop regions of the protein. The individual mutant constructs were cloned into a custom-made MSCV-based retroviral vector carrying an IRES-driven PuromycinR-T2A-mCherry double-selection. The library was provided as 204 individual plasmids.

For additional site-directed mutagenesis, AccuPrime Pfx DNA polymerase (Invitrogen) was used following the QuickChange II Site-directed Mutagenesis protocol from Agilent Technologies. The following MSCV-based retroviral vectors were used to express TLR7 and TLR9 in cell lines: MSCV2.2 (IRES-GFP), MSCV-Thy1.1 (IRES-Thy1.1), or MIGR2 (IRES-hCD2). TLR7 and TLR9 were fused to HA (YPYDVPDYA (SEQ ID NO: 11)) at the C-terminal end. TLR7 sequence was synthesized after codon optimization by Invitrogen’s GeneArt Gene Synthesis service and methods in the art.

### Cells and Tissue Culture Conditions

HEK293T (from ATCC) and GP2-293 packaging cell lines (Clontech) were cultured in DMEM complete media supplemented with 10% (vol/vol) FCS, L-glutamine, penicillin-streptomycin, sodium pyruvate, and HEPES (pH 7.2) (Invitrogen). RAW264 macrophage cell lines (ATCC) were cultured in RPMI 1640 (same supplements as above). BMMs were differentiated for seven days in RPMI complete media (same supplements as above plus 0.00034% (vol/vol) beta-mercaptoethanol) and supplemented with 10% (vol/vol) M-CSF containing supernatant from 3T3-CSF cells. BM-DC were differentiated for seven days in RPMI complete media (same supplements as above plus 0.00034% (vol/vol) beta-mercaptoethanol) and supplemented with 2% (vol/vol) GM-CSF containing supernatant from J558L cells.

To generate HEK293T Unc93b1^{-/-} cells, guide RNAs were designed and synthesized as gBlocks using methods in the art and then were subcloned into pUC19 (guide RNA: CTCACCTACGGCGTCTACC (SEQ ID NO: 12)). Humanized Cas9-2xNLS-GFP was a gift from the Doudna laboratory, University of California, Berkeley, CA. HEK293T cells were transfected using Lipofectamine LTX with equal amounts of the guide RNA plasmid and Cas9 plasmid. Seven days post transfection cells were plated in a limiting-dilution to obtain single cells. Correct targeting was verified by PCR analysis and loss of response to TLR9 and TLR7 stimulation in an NFkB luciferase assay. Unc93b1^{-/-} RAW macrophages were generated with the Cas9(D10A)-GFP nickase (guide RNAs: 1) GGCGCTTGCGGCGGTAGTAGCGG (SEQ ID NO: 13), 2) CGGAGTGGTCAAGAACGTGCTGG (SEQ ID NO: 14), 3) TTCGGAATGCGCGGCTGCCGCGG (SEQ ID NO: 15), 4) AGTCCGCGGCTACCGCTACCTGG (SEQ ID NO: 16)). Macrophages were transfected with Cas9 (D10A) and all four guide RNAs using Lipofectamine LTX and Plus reagent and single cell-sorted on Cas9-GFP two days later. Correct targeting was verified by loss of response to TLR7 stimulation and sequencing of the targeted region after TOPO cloning. Myd88 was knocked out in Unc93b1^{-/-} RAW macrophages stably expressing TLR7-HA and either Unc93b1^{WT} or Unc93b1^{PKP}. Cas9 transfection and screening of cells was performed as before, except for using Cas9-2xNLS-GFP (guide RNA: GGTTCAAGAACAGCGATAGG (SEQ ID NO: 17)).

### Retroviral Transduction

Retroviral transduction of RAW macrophages was performed using methods in the art. For macrophages expressing the Unc93b1 mutant library, transduced cells were selected with puromycin starting 48 hours after transduction and the efficiency of drug selection was verified by equal mCherry expression of target cells. When necessary, target cells were sorted on a Becton Dickinson Aria Fusion Sorter to match Unc93b1 expression levels using the bicistronic fluorescent reporter. For retroviral transduction of bone marrow derived macrophages, bone marrow was harvested and cultured in M-CSF-containing RPMI for two days. Progenitor cells were transduced with viral supernatant (produced as above) on two successive days by spinfection for 90 minutes at 32° C. 48 hours after the second transduction cells were put on Puromycin selection and cultured in M-CSF-containing RPMI media until harvested on Day 8.

### Pulse-Chase

Cells were seeded into 6 cm dishes the day before. After washing in PBS, cells were starved for 1 h in cysteine/methionine-free media (Corning) containing 10% dialyzed serum (dialyzed in PBS for two days using a 10 kD Snakeskin), then pulsed with 0.25 mCi/ml ³⁵S-cysteine/methionine (EasyTag Express Protei Labeling Mix, Perkin-Elmer). After a 45-min pulse, cells were washed and cultured in 5 ml chase media containing 0.45 mg/ml L-cysteine and L-methionine or harvested as the zero time point. Time points were harvested as follows: cells were washed twice in 2 ml PBS, then scraped in PBS and cell pellets were subjected to HA immunoprecipitation.

### Cell Fractionation by Sucrose Density Centrifugation

Cells in four confluent 15 cm dishes were washed in ice-cold PBS, scraped in 10 ml sucrose homogenization buffer (SHB: 250 µM sucrose, 3 mM imidazole pH 7.4) and pelleted by centrifugation. Cells were resuspended in 2 ml SHB plus protease inhibitor cocktail with EDTA (Roche) and 1 mM PMSF and disrupted by 25 strokes in a steel dounce homogenizer. The disrupted cells were centrifuged for 10 minutes at 1000 g to remove nuclei. Supernatants were loaded onto continuous sucrose gradients (percent iodixanol: 0, 10, 20, 30) and ultracentrifuged in an SW41 rotor at 25800 rpm for 2 h (Optima L-90K Ultracentrifuge, Beckman Coulter). 22 fractions of 420 µl were collected from top to bottom. 100 µl of each fraction were denatured in SDS buffer for western blot analysis. For immunoprecipitations, three fractions corresponding to ER or endosomes were combined and lysed for 1 hour after addition of protease inhibitor cocktail and NP-40 to a final concentration of 1%. Coimmunoprecipitation with anti-HA matrix was performed as described below.

### Exosome Purification

Exosomes were purified using methods in the art. Briefly, RAW macrophages were grown in 4 × 15 cm dishes, and 24 hours before exosome harvest the cell culture medium was replaced with exosome-depleted medium (RPMI 1640 + 10% FCS + supplements ultra-centrifuged overnight at 100,000 g). The next day cell supernatants were harvested, pooled (80 ml total), and subjected to sequential centrifugation steps at 4° C.: 1) 10 minutes at 300 g to remove live cells; 2) 20 minutes at 2,000 g to remove dead cells; 3) 30 minutes at 10,000 g to remove debris; and 4) 70 minutes at 100,000 g to pellet exosomes. Spins 3 and 4 were performed in an Optima L-90K Ultracentrifuge (Beckman Coulter) using an SW41 swinging-bucket rotor and 12 ml sample tubes. Exosomes were washed in PBS and centrifuged for another 60 minutes at 100,000 g. Final exosome pellets were lysed in 50-70 µl PBS + 1%NP-40 + Roche complete protease inhibitor cocktail for 30 minutes and then denatured in SDS loading buffer at room temperature for 1 hour. For comparison of exosome protein contents to whole cell lysates, some cells from the initial culture plates were lysed in NP-40 buffer (50 mM Tris [pH 7.4], 150 mM NaCl, 1% NP-40, 5 mM EDTA, supplemented with Roche complete protease inhibitor cocktail) for 1 hour at 4° C., centrifuged at maximum speed for 30 minutes at 4° C., and then denatured in SDS loading buffer at room temperature for 1 hour. 20 µl of cell and exosome lysates were kept for protein quantification with the Micro BCA Protein Assay Kit (Thermo Fisher). Between 5-10 µg of total protein was loaded per lane for western blot analysis.

### Luciferase Assays

Activation of NF-κB in HEK293T cells was performed using methods in the art. Briefly, transfections were performed in OptiMEM-I (Invitrogen) with LTX transfection reagent (Invitrogen) according to manufacturer’s guidelines. Cells were stimulated with CpG-B (200 nM - 1 µM), R848 (100-200 ng/ml), or human IL-1b (20 ng/ml) after 24 hours and lysed by passive lysis after an additional 12-16 hours. Luciferase activity was measured on an LMaxII-384 luminometer (Molecular Devices).

### Immunoprecipitation, Western Blot, and Dot Blot

Cells were lysed in NP-40 buffer (50 mM Tris [pH 7.4], 150 mM NaCl, 0.5% NP-40, 5 mM EDTA, supplemented with 1 mM PMSF, Roche complete protease inhibitor cocktail and PhosSTOP tablets). For ubiquitin blots, 40 mM N-Ethylmaleimide (Sigma) was added to the lysis buffer. After incubation at 4° C. for 1 hour, lysates were cleared of insoluble material by centrifugation. For immunoprecipitations, lysates were incubated with anti-HA matrix or anti-FLAG matrix (both pre-blocked with 1% BSA-PBS) for at least 2 hours, and washed four times in lysis buffer. Precipitated proteins were eluted in lysis buffer containing 200 ng/ml HA or 3xFLAG peptide, or denatured in SDS loading buffer at room temperature for 1 hour. Proteins were separated by SDS-PAGE (Bio-Rad TGX precast gels) and transferred to Immobilon PVDF membranes (Millipore) in a Trans-Blot Turbo transfer system (Bio-Rad). Membranes were blocked with Odyssey blocking buffer, probed with the indicated antibodies and developed using the Licor Odyssey Blot Imager. For dot blot: diluted peptides were dropwise added to nitrocellulose blotting membranes (GE Healthware). Membranes were dried at room temperature, blocked, and probed using the Licor Odyssey blot system.

Cell lysis and co-immunoprecipitations for Myddosome analyses were performed in the following buffer: 50 mM Tris-HCl pH 7.4, 150 mM NaCl, 10% glycerol, 1% NP-40 and supplemented with EDTA-free complete protease inhibitor cocktail (Roche), PhosSTOP (Roche) and 1 mM PMSF. Lysates were incubated overnight with anti-Myd88 antibody at 4° C., and then Protein G agarose (pre-blocked with 1% BSA-PBS) was added for additional 2 hours. Beads were washed four times in lysis buffer, incubated in SDS loading buffer at room temperature for 1 hour, separated by SDS-PAGE, and probed with the indicated antibodies.

### Tissue Harvest

Spleens and lymph nodes were digested with collagenase XI and DNase I for 30 minutes and single cell suspensions were generated by mechanical disruption. Red blood cells were lysed in ACK Lysing Buffer (Gibco).

### Flow Cytometry

Cells were seeded into non-treated tissue culture 24-well plates or round-bottom 96-well plates. The next day cells were stimulated with the indicated TLR ligands. To measure TNFα production, BrefeldinA (BD GolgiPlug, BD Biosciences) was added to cells 30 minutes after stimulation, and cells were collected after an additional 5.5 hours. Dead cells were excluded using a fixable live/dead stain (Violet fluorescent reactive dye, Invitrogen). Cells were stained for intracellular TNFα with a Fixation & Permeabilization kit according to manufacturer’s instructions (eBioscience).

For flow cytometry on mouse cells, dead cells were excluded using a fixable live/dead stain (Aqua fluorescent reactive dye, Invitrogen) or DAPI and all stains were carried out in PBS containing 1% BSA (w/v) and 0.1% Azide (w/v) including anti-CD16/32 blocking antibody. Cells were stained for 20 minutes at 4° C. with surface antibodies. Data were acquired on a LSRFortessa or X20 analyzer (BD Biosciences).

### Enzyme-Linked Immunosorbent Assay (ELISA) and Cytometric Bead Array (CBA)

Cells were seeded at 10⁵ cells/well into tissue culture-treated flat-bottom 96-well plates. The next day cells were stimulated with the indicated TLR ligands. For TNFα ELISAs, NUNC Maxisorp plates were coated with anti-TNFα at 1.5 µg/ml overnight at 4° C. Plates were then blocked with PBS + 1% BSA (w/v) at 37° C. for 1 hour before cell supernatants diluted in PBS + 1% BSA (w/v) were added and incubated at room temperature for 2 hours. Secondary anti-TNFα-biotin was used at 1 µg/ml followed by Streptavidin-HRP. Plates were developed with 1 mg/mL OPD in Citrate Buffer (PBS with 0.05 M NaH₂PO₄ and 0.02 M Citric acid, pH 5.0) with HCl acid stop.

For CBA, cell supernatants were collected as above and analyzed using the Mouse Inflammation Kit (BD Biosciences) according to the manufacturer’s instructions.

### Type I Interferon Production by BM-DCs

BM-DCs were seeded at 10⁵ cells/well into tissue culture-treated flat-bottom 96-well plates. The next day cells were stimulated with the indicated TLR ligands for 16 hours. The following day, supernatants were transferred onto L-292 ISRE-luciferase reporter cells to determine the amount of released type I IFN. Recombinant mouse IFN-β (pbl interferon source) was used for the standard curve. Reporter cells were incubated in BM-DC supernatants for 8 hours, lysed by passive lysis (Promega) and luciferase activity was measured on an LMaxII-384 luminometer (Molecular Devices).

### B Cell Proliferation Assay

Spleens were digested with collagenase 8 (Sigma) and DNAse-I for 45 minutes and red blood cells were lysed using ACK buffer (Gibco). Splenocytes were labeled with 12.5 µg/mL CFSE (Invitrogen) for 10 minutes at 37° C. and immediately underlayed with 3 ml FCS to spin out CSFE. Cells were taken up in media (RPMI/10%FCS/L-glutamine/Pen-Strep/HEPES/Sodium pyruvate/β-mercaptoethanol), counted, and seeded at 200,000 cells per well in round-bottom 96-well plates. Cells were incubated in media with various concentrations of CpG-B, R848, or LPS for 72 hours. Flow cytometry was used to analyze stimulated cells. Live, singlet cells were pre-gated on CD19⁺ and cell proliferation was determined by the geometric mean fluorescence intensity (gMFI) of CFSE. For the quantification, a proliferation index was determined by dividing the gMFI CSFE of the unstimulated control by the gMFI CSFE of the stimulated sample (CSFE^{Unstim}:CFSE^{Sample}) and plotted along the ligand titration.

### ANA Staining

Mouse sera were diluted 1:80 in 1% BSA-PBS and applied to MBL Bion Hep-2 antigen substrate IF A test system for 1 hour at room temperature. Slides were washed 3 times with PBS and incubated for 30 minutes with a mixture of fluorophore-conjugated secondary antibodies against anti-mouse IgG and IgM. Slides were washed 3 times and incubated with DAPI for 5 minutes. After rinsing once with PBS, slides were mounted in VectaShield Hard Set, and imaged on a Zeiss AxioZoom Z.1 slide scanner.

### Microscopy

Cells were plated onto coverslips and allowed to settle overnight. Coverslips were washed with PBS, fixed with 4% PFA-PBS for 15 minutes, and permeabilized with 0.5% saponin-PBS for 5 minutes. To quench PFA autofluorescence coverslips were treated with sodium borohydride/0.1% saponin-PBS for 10 minutes. After washing 3x with PBS, cells were blocked in 1% BSA/0.1% saponin-PBS for 1 hour. Slides were stained in blocking buffer with anti-HA and anti-LAMP1 (see antibodies above), washed with PBS and incubated for 45 minutes with secondary antibodies. Cells were washed 3x in PBS and mounted in VectaShield Hard Set. Cells were imaged on a Zeiss Elyra PS.1 with a 100x/1.46 oil immersion objective in Immersol 518F / 30° C. (Zeiss). Z-Sections were acquired, with three grid rotations at each Z-position. The resulting dataset was SIM processed and Channel Aligned using Zeiss default settings in Zen. The completed super-resolution Z-Series was visualized and analyzed using Fiji and methods in the art. To compare the degree of colocalization of two proteins a single section from the middle of the Z-Series was selected and analyzed using a customized pipeline for object-based colocalization in Cell Profiler and methods in the art. Briefly, primary objects (TLR7 vs Lamp1, or Unc93b1 vs Lamp1) were identified and related to each other to determine the degree of overlap between objects. Data are expressed as % of object 1 colocalized with object 2.

### Phagosome Isolation and Protein Complex Purification

Cells in a confluent 15 cm dish were incubated with about 10⁸ 1 µm magnetic beads (Polysciences) for 4 hours. After rigorous washing in PBS, cells were scraped into 10 ml sucrose homogenization buffer (SHB: 250 µM sucrose, 3 mM imidazole, pH 7.4) and pelleted by centrifugation. Cells were resuspended in 2 ml SHB plus protease inhibitor cocktail with EDTA (Roche) and 1 mM PMSF and disrupted by 25 strokes in a steel dounce homogenizer. The disrupted cells were gently rocked for 10 minutes on ice to free endosomes. Beads were collected with a magnet (Dynal) and washed 4x with SHB plus protease inhibitor. After the final wash, phagosome preparations were denatured in 2x SDS buffer at room temperature for 1 hour and analyzed by western blot.

For protein complex purification, phagosome preparations were lysed in NP-40 buffer (50 mM Tris, pH 7.4, 150 mM NaCl, 0.5% NP-40, 5 mM EDTA, supplemented with 1 mM PMSF, complete protease inhibitor cocktail and PhosSTOP tablets (Roche) on ice for 1 hour. Magnetic beads were removed by magnet and insoluble components were precipitated by 15,000 g spin for 20 minutes. Lysate was incubated with anti-FLAG matrix for 3 hours, followed by four washes in lysis buffer. Proteins were eluted in NP-40 buffer containing 200 ng/ml 3xFLAG peptide, and were further applied to western blot, silver stain or Trypsin in-solution digest for mass spectrometry.

### Mass Spectrometry

Proteins were simultaneously extracted from a gel slice and digested with trypsin, and the resulting peptides were dried and resuspended in buffer A (5% acetonitrile/ 0.02% heptaflurobutyric acid (HBFA)). A nano LC column that consisted of 10 cm of Polaris c18 5 µm packing material (Varian) was packed in a 100 µm inner diameter glass capillary with an emitter tip. After sample loading and washed extensively with buffer A, the column was then directly coupled to an electrospray ionization source mounted on a Thermo-Fisher LTQ XL linear ion trap mass spectrometer. An Agilent 1200 HPLC equipped with a split line so as to deliver a flow rate of 300 nl/min was used for chromatography. Peptides were eluted using a 90 minute gradient from buffer A to 60% Buffer B (80% acetonitrile/ 0.02% HBFA).

Protein identification and quantification were done with IntegratedProteomics Pipeline (IP2, Integrated Proteomics Applications, Inc. San Diego, CA) using ProLuCID/Sequest, DTASelect2 and Census. Tandem mass spectra were extracted from raw files using RawExtractor and were searched against the mouse protein database (obtained from UNIPROT) plus sequences of common contaminants, concatenated to a decoy database in which the sequence for each entry in the original database was reversed. LTQ data was searched with 3000.0 milli-amu precursor tolerance and the fragment ions were restricted to a 600.0 ppm tolerance. All searches were parallelized and searched on the VJC proteomics cluster. Search space included all fully tryptic peptide candidates with no missed cleavage restrictions. Carbamidomethylation (+57.02146) of cysteine was considered a static modification. One peptide per protein and both tryptic termini was used for each peptide identification. The ProLuCID search results were assembled and filtered using the DTASelect program with a peptide false discovery rate (FDR) of 0.001 for single peptides and a peptide FDR of 0.005 for additional peptides for the same protein. Under such filtering conditions, the estimated false discovery rate was zero for the datasets used.

### Quantification and Statistical Analysis

Statistical parameters, including the exact value of n and statistical significance, are reported in the Figures and Figure Legends, whereby n refers to the number of repeats within the same experiment. Representative images have been repeated at least three times, unless otherwise stated in the figure legends. Data is judged to be statistically significant when p < 0.05 by Student’s t-test. To compare the means of several independent groups, a one-way ANOVA followed by a Tukey’s posttest was used. To compare means of different groups across a dose response, a two-way ANOVA followed by a Bonferroni posttest was used. In figures, asterisks denote statistical significance (*, p < 0.05; **, p < 0.01; ***, p < 0.001). Statistical analysis was performed in GraphPad PRISM 7 (Graph Pad Software Inc.).

### REFERENCES

The following references are herein incorporated by reference in their entirety with the exception that, should the scope and meaning of a term conflict with a definition explicitly set forth herein, the definition explicitly set forth herein controls:

Al-Mayouf, SM, et al. Loss-of-function variant in DNASE1L3 causes a familial form of systemic lupus erythematosus. Nat Genet 43, 1186-1188, doi: 10.1038/ng.975 (2011).

Baietti, MF, et al. Syndecan-syntenin-ALIX regulates the biogenesis of exosomes. Nat Cell Biol 14, 677-685, doi: 10.1038/ncb2502 (2012).

Barton, GM, et al. Intracellular localization of Toll-like receptor 9 prevents recognition of self DNA but facilitates access to viral DNA. Nat Immunol 7, 49-56, doi: 10.1038/ni1280 (2006).

Baum, R, et al. Cutting edge: AIM2 and endosomal TLRs differentially regulate arthritis and autoantibody production in DNase II-deficient mice. J Immunol 194, 873-877, doi: 10.4049/jimmunol.1402573 (2015).

Brinkmann, MM, et al. The interaction between the ER membrane protein UNC93B and TLR3, 7, and 9 is crucial for TLR signaling. J Cell Biol 177, 265-275, doi: 10.1083/jcb.200612056 (2007).

Carpenter, AE, et al. CellProfiler: image analysis software for identifying and quantifying cell phenotypes. Genome Biol 7, R100, doi: 10.1186/gb-2006-7-10-r100 (2006).

Casrouge, A, et al. Herpes simplex virus encephalitis in human UNC-93B deficiency. Science 314, 308-312, doi: 10.1126/science.1128346 (2006).

Chen, F, et al. Syntenin negatively regulates TRAF6-mediated IL-1R/TLR4 signaling. Cell Signal 20, 666-674, doi: 10.1016/j.cellsig.2007.12.002 (2008).

Christensen, SR, et al. Toll-like receptor 7 and TLR9 dictate autoantibody specificity and have opposing inflammatory and regulatory roles in a murine model of lupus. Immunity 25, 417-428, doi: 10.1016/j.immuni.2006.07.013 (2006).

Ewald, SE, et al. The ectodomain of Toll-like receptor 9 is cleaved to generate a functional receptor. Nature 456, 658-662, doi: 10.1038/nature07405 (2008).

Fukui, R, et al. Unc93B1 biases Toll-like receptor responses to nucleic acid in dendritic cells toward DNA- but against RNA-sensing. J Exp Med 206, 1339-1350, doi: 10.1084/jem.20082316 (2009).

Fukui, R, et al. Unc93B1 restricts systemic lethal inflammation by orchestrating Toll-like receptor 7 and 9 trafficking. Immunity 35, 69-81, doi: 10.1016/j.immuni.2011.05.010 (2011).

Garcia-Cattaneo, A, et al. Cleavage of Toll-like receptor 3 by cathepsins B and H is essential for signaling. PNAS USA 109, 9053-9058, doi: 10.1073/pnas.1115091109 (2012).

Ghossoub, R, et al. Syntenin-ALIX exosome biogenesis and budding into multivesicular bodies are controlled by ARF6 and PLD2. Nat Commun 5, 3477, doi: 10.1038/ncomms4477 (2014).

Grassel, L, et al. The CD63-Syntenin-1 Complex Controls Post-Endocytic Trafficking of Oncogenic Human Papillomaviruses. Sci Rep 6, 32337, doi: 10.1038/srep32337 (2016). https: //www.phosphosite.org/proteinAction.action?id=14824.

Huh, JW, et al. UNC93B1 is essential for the plasma membrane localization and signaling of Toll-like receptor 5. PNAS USA 111, 7072-7077, doi: 10.1073/pnas.1322838111 (2014).

Jeppesen, DK, et al. Reassessment of Exosome Composition. Cell 177, 428-445 e418, doi: 10.1016/j.cell.2019.02.029 (2019).

Kagan & Barton. Emerging principles governing signal transduction by pattern-recognition receptors. Cold Spring Harb Perspect Biol 7, a016253, doi: 10.1101/cshperspect.a016253 (2014).

Kegelman, TP, et al. Targeting tumor invasion: the roles of MDA-9/Syntenin. Expert Opin Ther Targets 19, 97-112, doi: 10.1517/14728222.2014.959495 (2015).

Kim, YM, et al. UNC93B1 delivers nucleotide-sensing toll-like receptors to endolysosomes. Nature 452, 234-238, doi: 10.1038/nature06726 (2008).

Latty, SL, et al. Activation of Toll-like receptors nucleates assembly of the MyDDosome signaling hub. Elife 7, doi: 10.7554/eLife.31377 (2018).

Lee, BL, et al. UNC93B1 mediates differential trafficking of endosomal TLRs. Elife 2, e00291, doi: 10.7554/eLife.00291 (2013).

Majer, O, et al. Nucleic acid-sensing TLRs: trafficking and regulation. Curr Opin Immunol 44, 26-33, doi: 10.1016/j.coi.2016.10.003 (2017).

Majer, O, et al. Release from Unc93b1 in endosomes reinforces the compartmentalized activation of select nucleic acid-sensing Toll-like receptors. Submitted.

Mali, P, et al. RNA-guided human genome engineering via Cas9. Science 339, 823-826, doi: 10.1126/science.1232033 (2013).

Napirei, M, et al. Features of systemic lupus erythematosus in Dnase1-deficient mice. Nat Genet 25, 177-181, doi: 10.1038/76032 (2000).

Nickerson, KM, et al. TLR9 regulates TLR7- and MyD88-dependent autoantibody production and disease in a murine model of lupus. J Immunol 184, 1840-1848, doi: 10.4049/jimmunol.0902592 (2010).

Park, B, et al. Proteolytic cleavage in an endolysosomal compartment is required for activation of Toll-like receptor 9. Nat Immunol 9, 1407-1414, doi: 10.1038/ni.1669 (2008).

Pisitkun, P, et al. Autoreactive B cell responses to RNA-related antigens due to TLR7 gene duplication. Science 312, 1669-1672, doi: 10.1126/science.1124978 (2006).

Raiborg & Stenmark. The ESCRT machinery in endosomal sorting of ubiquitylated membrane proteins. Nature 458, 445-452, doi: 10.1038/nature07961 (2009).

Roberts, AW, et al. Tissue-Resident Macrophages Are Locally Programmed for Silent Clearance of Apoptotic Cells. Immunity 47, 913-927 e916, doi: 10.1016/j.immuni.2017.10.006 (2017).

Schindelin, J, et al. Fiji: an open-source platform for biological-image analysis. Nat Methods 9, 676-682, doi: 10.1038/nmeth.2019 (2012).

Shields & Piper. How ubiquitin functions with ESCRTs. Traffic 12, 1306-1317, doi: 10.1111/j.1600-0854.2011.01242.x (2011).

Sisirak, V, et al. Digestion of Chromatin in Apoptotic Cell Microparticles Prevents Autoimmunity. Cell 166, 88-101, doi: 10.1016/j.cell.2016.05.034 (2016).

Sorkin & von Zastrow. Endocytosis and signalling: intertwining molecular networks. Nat Rev Mol Cell Biol 10, 609-622, doi: 10.1038/nrm2748 (2009).

Subramanian, S, et al. A Tlr7 translocation accelerates systemic autoimmunity in murine lupus. PNAS USA 103, 9970-9975, doi: 10.1073/pnas.0603912103 (2006).

Tabeta, K, et al. The Unc93b1 mutation 3d disrupts exogenous antigen presentation and signaling via Toll-like receptors 3, 7 and 9. Nat Immunol 7, 156-164, doi: 10.1038/ni1297 (2006).

Taelman, VF, et al. Wnt signaling requires sequestration of glycogen synthase kinase 3 inside multivesicular endosomes. Cell 143, 1136-1148, doi: 10.1016/j.cell.2010.11.034 (2010).

Tamura, K, et al. Increased production of intestinal immunoglobulins in Syntenin-1-deficient mice. Immunobiology 220, 597-604, doi: 10.1016/j.imbio.2014.12.003 (2015).

Thery, C, et al. A. Isolation and characterization of exosomes from cell culture supernatants and biological fluids. Curr Protoc Cell Biol Chapter 3, Unit 3 22, doi: 10.1002/0471143030.cb0322s30 (2006).

Tsokos, GC, et al. New insights into the immunopathogenesis of systemic lupus erythematosus. Nat Rev Rheumatol 12, 716-730, doi: 10.1038/nrrheum.2016.186 (2016).

Yasutomo, K, et al. Mutation of DNASE1 in people with systemic lupus erythematosus. Nat Genet 28, 313-314, doi: 10.1038/91070 (2001).

Akilesh HM, et al. Chronic TLR7 and TLR9 signaling drives anemia via differentiation of specialized hemophagocytes. Science. 2019 Jan 11;363(6423). pii: eaao5213. doi: 10.1126/science.aao5213.

Arpaia. N, et al. A Distinct Function of Regulatory T Cells in Tissue Protection Cell. 162(5): 1078-1089, doi: 10.1016/j.cell.2015.08.021 (2015).

Baccarella A, et al. Toll-like receptor 7 mediates early innate immune responses to malaria. Infect Immun. 2013 Dec;81(12): 4431-42. doi: 10.1128/IAI.00923-13.

Balak DM, et al. IMO-8400, a toll-like receptor 7, 8, and 9 antagonist, demonstrates clinical activity in a phase 2a, randomized, placebo-controlled trial in patients with moderate-to-severe plaque psoriasis. Clin Immunol. 2017 Jan;174: 63-72. doi: 10.1016/j.clim.2016.09.015.

Burzyn, D, et al. A special population of regulatory T cells potentiates muscle repair. Cell. 155(6): 1282-95. doi: 10.1016/j.cell.2013.10.054 (2013).

Cheadle EJ, et al. A TLR7 agonist enhances the antitumor efficacy of obinutuzumab in murine lymphoma models via NK cells and CD4 T cells. Leukemia. 2017 Oct;31(10): 2278. doi: 10.1038/leu.2017.218.

Deane, JA, et al. Control of toll-like receptor 7 expression is essential to restrict autoimmunity and dendritic cell proliferation. Immunity 27, 801-810, doi: 10.1016/j.immuni.2007.09.009 (2007).

Du K, et al. Recent advances in the discovery and development of TLR ligands as novel therapeutics for chronic HBV and HIV infections. Expert Opin Drug Discov. 2018 Jul;13(7): 661-670. doi: 10.1080/17460441.2018.1473372.

Funk E, et al. Tickling the TLR7 to cure viral hepatitis. J Transl Med. 2014 May 14;12: 129. doi: 10.1186/1479-5876-12-129.

Goff PH, et al. Synthetic Toll-like receptor 4 (TLR4) and TLR7 ligands as influenza virus vaccine adjuvants induce rapid, sustained, and broadly protective responses. J Virol. 2015 Mar;89(6): 3221-35. doi: 10.1128/JVI.03337-14.

Guiducci, C, et al. RNA recognition by human TLR8 can lead to autoimmune inflammation. J Exp Med. 210(13): 2903-2919. doi: 10.1084/jem.20131044 (2013).

Hayashi T, et al. Treatment of autoimmune inflammation by a TLR7 ligand regulating the innate immune system. PLoS One. 2012;7(9): e45860. doi: 10.1371/journal.pone.0045860.

Hosoya T, et al. Induction of oligoclonal CD8 T cell responses against pulmonary metastatic cancer by a phospholipid-conjugated TLR7 agonist. PNAS USA. 2018;115(29): E6836-E6844. doi: 10.1073/pnas.1803281115.

Hwang SH, et al. B cell TLR7 expression drives anti-RNA autoantibody production and exacerbates disease in systemic lupus erythematosus-prone mice. J Immunol. 2012;189(12): 5786-5796. doi: 10.4049/jimmunol.1202195.

Jurk, M, et al. Human TLR7 or TLR8 independently confer responsiveness to the antiviral compound R-848. Nat Immunol. 3(6): 499. doi: 10.1038/ni0602-499 (2002).

Khan, AR, et al. Ligation of TLR7 on CD19+CD1dhi B cells suppresses allergic lung inflammation via T regulatory cells. European journal of immunology. 2015; 45. doi: 10.1002/eji.201445211.

Kim KW, et al. Toll-like receptor 7 regulates osteoclastogenesis in rheumatoid arthritis. J Biochem. 2019 Sep 1;166(3): 259-270. doi: 10.1093/jb/mvz033.

Kranz LM, et al. Systemic RNA delivery to dendritic cells exploits antiviral defence for cancer immunotherapy. Nature. 2016 Jun 16;534(7607): 396-401. doi: 10.1038/nature18300.

Lalive PH, et al. TLR7 signaling exacerbates CNS autoimmunity through downregulation of Foxp3+ Treg cells. Eur J Immunol. 2014 Jan;44(1): 46-57. doi: 10.1002/eji.201242985.

Lam, AJ, et al. Innate Control of Tissue-Reparative Human Regulatory T Cells. J Immunol. 202(8): 2195-2209. doi: 10.4049/jimmunol.1801330 (2019).

Macal M, et al. Plasmacytoid dendritic cells are productively infected and activated through TLR-7 early after arenavirus infection. Cell Host Microbe. 2012;11(6): 617-630. doi: 10.1016/j.chom.2012.04.017.

Misiak A, et al. Addition of a TLR7 agonist to an acellular pertussis vaccine enhances Th1 and Th17 responses and protective immunity in a mouse model. Vaccine. 2017 Sep 18;35(39): 5256-5263. doi: 10.1016/j.vaccine.2017.08.009.

Nazmi A, et al. TLR7 is a key regulator of innate immunity against Japanese encephalitis virus infection. Neurobiol Dis. 2014 Sep;69: 235-47. doi: 10.1016/j.nbd.2014.05.036.

Neighbours LM, et al. Myd88-dependent toll-like receptor 7 signaling mediates protection from severe Ross River virus-induced disease in mice. J Virol. 2012;86(19): 10675-10685. doi: 10.1128/JVI.00601-12.

Nian, H, et al. R-848 triggers the expression of TLR⅞ and suppresses HIV replication in monocytes. BMC Infect Dis 12, 5 (2012) doi: 10.1186/1471-2334-12-5.

Okazaki S, et al. Predictive value of TLR7 polymorphism for cetuximab-based chemotherapy in patients with metastatic colorectal cancer. Int J Cancer. 2017;141(6): 1222-1230. doi: 10.1002/ijc.30810.

Roach, JC, et al. The evolution of vertebrate Toll-like receptors. PNAS USA 102(27): 9577-82, doi: 10.1073/pnas.0502272102 (2005).

Souyris M, et al. TLR7 escapes X chromosome inactivation in immune cells. Sci Immunol. 2018 Jan 26;3(19). pii: eaap8855. doi: 10.1126/sciimmunol.aap8855.

Town T, et al. Toll-like receptor 7 mitigates lethal West Nile encephalitis via interleukin 23-dependent immune cell infiltration and homing. Immunity. 2009;30(2): 242-253. doi: 10.1016/j.immuni.2008.11.012.

Van LP, et al. Treatment with the TLR7 agonist R848 induces regulatory T-cell-mediated suppression of established asthma symptoms. Eur J Immunol. 2011 Jul;41(7): 1992-9. doi: 10.1002/eji.201040914.

Vascotto F, et al. Intravenous delivery of the toll-like receptor 7 agonist SC1 confers tumor control by inducing a CD8+ T cell response. Oncoimmunology. 2019 Apr 19;8(7): 1601480. doi: 10.1080/2162402X.2019.1601480.

Vo HTM, et al. Alum/Toll-Like Receptor 7 Adjuvant Enhances the Expansion of Memory B Cell Compartment Within the Draining Lymph Node. Front Immunol. 2018;9: 641. Published 2018 Apr 9. doi: 10.3389/fimmu.2018.00641.

Xie G, et al. Dysregulation of Toll-Like Receptor 7 Compromises Innate and Adaptive T Cell Responses and Host Resistance to an Attenuated West Nile Virus Infection in Old Mice. J Virol. 2015;90(3): 1333-1344. Published 2015 Nov 18. doi: 10.1128/JVI.02488-15.

Ye J, et al. TLR7 Signaling Regulates Th17 Cells and Autoimmunity: Novel Potential for Autoimmune Therapy. J Immunol. 2017;199(3): 941-954. doi: 10.4049/jimmunol.1601890.

Zhang M, et al. Toll-like receptors 7 and 8 expression correlates with the expression of immune biomarkers and positively predicts the clinical outcome of patients with melanoma. Onco Targets Ther. 2017;10: 4339-4346. Published 2017 Sep 1. doi: 10.2147/OTT.S136194.

Zhang, Z, et al. Structural Analysis Reveals that Toll-like Receptor 7 Is a Dual Receptor for Guanosine and Single-Stranded RNA. Immunity. 45(4): 737-748. doi: 10.1016/j.immuni.2016.09.011 (2016).

Hultquist JF, et al. CRISPR-Cas9 genome engineering of primary CD4⁺ T cells for the interrogation of HIV-host factor interactions. Nat Protoc. 2019;14(1):1-27. doi:10.1038/s41596-018-0069-7.

Morgan & Boyerinas, Genetic Modification of T Cells. Biomedicines. 2016;4(2):9. Published 2016 Apr 20. doi:10.3390/biomedicines4020009.

All scientific and technical terms used in this application have meanings commonly used in the art unless otherwise specified.

As used herein, the terms “protein”, “polypeptide” and “peptide” are used interchangeably to refer to two or more amino acids linked together.

Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′ .

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

As used herein, an amino acid or nucleotide of a given sequence that “corresponds” to an amino acid or nucleotide of a reference sequence refers to the amino acid or nucleotide of the given sequence that aligns with the amino acid or nucleotide of the reference sequence when the given sequence and the reference sequence are optimally aligned. Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human. In some embodiments, the subject is in need of toll-like receptor modulation. As used herein, a subject in need of toll-like receptor modulation is one who may likely benefit from (1) increasing trafficking or signaling of TLR7 and/or TLR8, or (2) decreasing trafficking or signaling of TLR7 and/or TLR8. Subjects in need of toll-like receptor modulation include those who exhibit abnormal levels of trafficking or signaling of TLR7 and/or TLR8.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A”.

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C”. As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C”.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A mutant Unc93b1 protein comprising at least one amino acid mutation as compared to its unmutated wildtype sequence, with the proviso that the at least one amino acid mutation does not correspond to D34A; Y99A; Y154A; K197A; H412R; PRQ(524,525,526)/AAA; PKP(530,531,532)/AAA; DNS(545,546,547)/AAA; S547A; DES(548,549,550)/AAA of SEQ ID NO: 1.

2. The mutant Unc93b1 protein according to claim 1, wherein, the at least one amino acid mutation corresponds to one or more mutations as set forth in Figure 1.

3. The mutant Unc93b1 protein according to claim 1, wherein
the unmutated wildtype sequence comprises at least 90% sequence identity to SEQ ID NO: 1 or SEQ ID NO: 2,
the at least one amino acid mutation corresponds to one of the mutations provided in Figure 1,
the amino acid sequence of the mutant Unc93b1 protein comprises less than 100% sequence identity to naturally occurring unc-93 homolog B1 proteins, and/or
the amino acid sequence of the mutant Unc93b1 protein comprises at least 85% sequence identity to SEQ ID NO: 1 or SEQ ID NO: 2.

4. A method of modulating the trafficking and/or signaling of a Toll-Like Receptor in a cell or subject, which comprises administering to the cell or subject one or more mutant Unc93b1 proteins according to claim 1.

5. The method according to claim 4, wherein the Toll-Like Receptor is Toll-Like Receptor 7 (TLR7) or a Toll-Like Receptor 8 (TLR8).

6. The method according to claim 5, wherein, compared to a negative control, the signaling of the Toll-Like Receptor is increased and the at least one amino acid mutation corresponds to one or more of the following mutations of SEQ ID NO: 1: EPL(30,32,33); DEL(34,35,36); EEEEE(45,46,47,48,49); YY(52,53); REV(95,96,97); LPD(104,105,106); IDS(107,108,109); K110; R157; YKE(196,197,198); QDE(199,200,201); Y191; Y196; IDL(317,318,319); QLP(329,330,331); RR(339,341); LRH(340,341,342); F352; P404; LQH(429,430,431); SWI(432,433,434); W477; K496; W513; KPK(531,532,535); QHK(533,534,535); VRG(536,537,538); LEE(542,543,544); DME(551,552,553); and S(187,212,432,547,550).

7. The method according to claim 5, wherein, compared to a negative control, the signaling of the Toll-Like Receptor is decreased and the at least one amino acid mutation corresponds to one or more of the following mutations of SEQ ID NO: 1: EVE(2,3,4); PP(6,9); Y8; VG(10,12); GPQ(15,16,17); GDE(18,19,20); DRH(21,22,23); GVP(24,25,26); DGP(27,28,29); VGY(37,38,40); RR(50,51); RR(54,55); KRL(56,57,58); Y75; Y78; QMQ(83,84,85); LIL(86,87,88); HYD(89,90,91); ETY(92,93,94); KYG(98,99,100); NMG(101,102,103); Y94; RK(95,98); YN(99,101); Y125; P127; F132; F133; GTK(134,135,136); WMM(137,138,139); F140; Y146; F149; W155; E156; YYT(158,159,160); T160; P163; P174; W176; TRM(184,185,186); SQK(187,188,189); YYE(190,191,192); YSH(193,194,195); QGP(202,203,204); S187; Y190; Y193; PP(208,209); RGS(210,211,212); HPY(213,215,216); F220; F224; Y225; F227; F228; H229; F232; P238; IYF(240,241,242); LNN(243,244,245); YLY(246,247,248); DLN(249,250,251); HTL(252,253,254); INV(255,256,257); QSC(258,259,260); GTK(261,262,263); SQG(264,265,266); ILN(267,268,269); GFN(270,271,272); KTV(273,274,275); LRT(276,277,278); LPR(279,280,281); SKN(282,283,284); F297; GAA(308,309,310); YRP(311,312,313); TEE(314,315,316); RSV(320,321,322); GWG(323,324,325); NIF(326,327,328); FKH(332,333,334); PE(313,315); RW(320,324); VRD(335,336,337); P345; F346; F347; Y349; F356; F361; Y365; GVC(366,367,368); SMG(369,370,371); LER(372,373,374); Y377; Y382; PR(426,427); F420; F421; W422; PRV(426,427,428); FYF(435,436,437); WF(433,437); W442; Y461; EDK(462,463,464); ERQ(465,466,467); DFI(468,469,470); FT(471,472); W476; Y486; MKK(493,494,496); K494; Y511; EQK(515,516,517); QQ(519,520); PP(524,527); PRI(527,528,529); PP(527,530); Y539; Y541; GEQ(554,555,556); GQG(557,558,559); DC(560,561); EDE(563,564,565); PQG(567,568,570); PLG(571,572,573); EPP(575,576,579); GPC(578,579,580); RKP(581,582,583); CPY(584,585,586); EQL(587,588,590); GGD(591,592,593); Y(8,40,52,53,94,99,158,159,190,191,193,196,541,586); S(187,212,432,547,550) + T(93,160,314); PP(5,6) + PP(6,9) + PPP(26,29,32) + YN(40,42); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and RR(50,51) + RRR(54,55,57) + RR(339,341).

8. The method according to claim 5, wherein, compared to a negative control, the trafficking of the Toll-Like Receptor is decreased and the at least one amino acid mutation corresponds to one or more of the following mutations of SEQ ID NO: 1: Y75; QMQ(83,84,85); LIL(86,87,88); HYD(89,90,91); ETY(92,93,94); KYG(98,99,100); Y94; RK(95,98); GTK(134,135,136); IYF(240,241,242); YLY(246,247,248); QSC(258,259,260); LRT(276,277,278); LPR(279,280,281); SKN(282,283,284); FKH(332,333,334); RW(320,324); VRD(335,336,337); F361; Y365; GVC(366,367,368); SMG(369,370,371); LER(372,373,374); W442; ERQ(465,466,467); MKK(493,494,496); EQK(515,516,517); PP(524,527); Y(8,40,52,53,94,99,158,159,190,191,193,196,541,586); S(187,212,432,547,550) + T(93,160,314); TY(93,94) + REK(95,96,98) + YN(99,101); YF(241,242) + YL(246,247); PE(313,315) + RW(320,324) + FF(328,332); PPP(524,527,530) + KPK(531,532,535) + Y541 + PP(576,579); and RR(50,51) + RRR(54,55,57) + RR(339,341).

9. The method according to claim 4, wherein a nucleic acid molecule encoding the one or more mutant Unc93b1 proteins is administered to the cell or subject.

10. The method according to claim 4, wherein a host cell that expresses the one or more mutant Unc93b1 proteins is administered to the subject.

11. The method according to claim 4, wherein the one or more mutant Unc93b1 proteins is administered by modifying a Unc93b1 gene of the cell or subject to express the one or more mutant Unc93b1 proteins, wherein the Unc93b1 gene is endogenous to the cell or subject.

12. The method according to claim 4, wherein the one or more mutant Unc93b1 proteins is administered in the form of a pharmaceutical composition.

13. The method according to claim 4, wherein the subject is in need of toll-like receptor modulation.

14. A nucleic acid molecule that encodes the mutant Unc93b1 protein according to claim 1.

15. A host cell comprising the mutant Unc93b1 protein according to claim 1 or a nucleic acid molecule encoding the mutant Unc93b1 protein.

16. A composition comprising (a) the mutant Unc93b1 protein according to claim 1, a nucleic acid molecule encoding the mutant Unc93b1 protein, and/or host cell comprising the mutant Unc93b1 protein or the nucleic acid molecule, and (b) a pharmaceutically acceptable carrier.

17. A kit comprising (a) the mutant Unc93b1 protein according to claim 1, a nucleic acid molecule encoding the mutant Unc93b1 protein, host cell comprising the mutant Unc93b1 protein or the nucleic acid molecule, and/or a composition thereof, (b) packaged together with a drug delivery device.

18. A method of inducing or increasing regulatory T cell proliferation, production of amphiregulin, and/or tissue repair in a subject, which comprises preforming the method according to claim 4.

19. A method of inducing or increasing CD8+ T cell proliferation, production of interferon-gamma, and/or a co-stimulatory immune response in a subject, which comprises preforming the method according to claim 4.


SEQUENCE LISTING (17 sequences)

SEQ ID NO: 1 - protein, 598 aa, Mus musculus
MEVEPPLYPVAGAAGPQGDEDRHGVPDGPEAPLDELVGAYPNYNEEEEERRYYRRKRLGVVKNVLAASTGVTLTYGVYLGLLQMQLILHYDETYREVKYGNMGLPDIDSKMLMGINVTPIAALLYTPVLIRFFGTKWMMFLAVGIYALFVSTNYWERYYTLVPSAVALGMAIVPLWASMGNYITRMSQKYYEYSHYKEQDEQGPQQRPPRGSHAPYLLVFQAIFYSFFHLSFACAQLPMIYFLNNYLYDLNHTLINVQSCGTKSQGILNGFNKTVLRTLPRSKNLIVVESVLMAVAFLAMLMVLGLCGAAYRPTEEIDLRSVGWGNIFQLPFKHVRDFRLRHLVPFFIYSGFEVLFACTGFALGYGVCSMGLERLAYLLIAYSLGASASSVLGLLGLWLPRSVPLVAGAGLHLLLTLSLFFWAPAPRVLQHSWIFYFVAALWGVGSALNKTGLSTLLGILYEDKERQDFIFTIYHWWQAVAIFVVYLGSSLPMKAKLAVLLVTLVAAAASYLWMEQKLQQGLVPRQPRIPKPQHKVRGYRYLEEDNSDESDMEGEQGQGDCAEDEAPQAGPLGAEPAGPCRKPCPYEQALGGDGPEEQ

SEQ ID NO: 2 - protein, 597 aa, Homo sapiens
MEAEPPLYPMAGAAGPQGDEDLLGVPDGPEAPLDELVGAYPNYNEEEEERRYYRRKRLGVLKNVLAASAGGMLTYGVYLGLLQMQLILHYDETYREVKYGNMGLPDIDSKMLMGINVTPIAALLYTPVLIRFFGTKWMMFLAVGIYALFVSTNYWERYYTLVPSAVALGMAIVPLWASMGNYITRMAQKYHEYSHYKEQDGQGMKQRPPRGSHAPYLLVFQAIFYSFFHLSFACAQLPMIYFLNHYLYDLNHTLYNVQSCGTNSHGILSGFNKTVLRTLPRSGNLIVVESVLMAVAFLAMLLVLGLCGAAYRPTEEIDLRSVGWGNIFQLPFKHVRDYRLRHLVPFFIYSGFEVLFACTGIALGYGVCSVGLERLAYLLVAYSLGASAASLLGLLGLWLPRPVPLVAGAGVHLLLTFILFFWAPVPRVLQHSWILYVAAALWGVGSALNKTGLSTLLGILYEDKERQDFIFTIYHWWQAVAIFTVYLGSSLHMKAKLAVLLVTLVAAAVSYLRMEQKLRRGVAPRQPRIPRPQHKVRGYRYLEEDNSDESDAEGEHGDGAEEEAPPAGPRPGPEPAGLGRRPCPYEQAQGGDGPEEQ

SEQ ID NO: 3 - protein, 623 aa, Danio rerio
MAALIADDNVYREAELDGAPPAEAEDPPQLPGPEGNIQGQVEEFLGPHPDYDEEEEERKYYRRKRLGVVKNVVGASCGGMIIYSVYMGLLQMQLILHYDETYREVKYGTLGLEDIDNKMLMGINVTPIVGLLYTPVLIRFLGTKWMMFLASGIYALFVSTNYWERYYTLVPSAVAIGVVIVPLWASMGNYITRMAQQYYEFVNYKDENVQEQKKPPKGAFHSYIIIFQSVFYIIFHLSFVFAEFPTVLFLNNYLNDYRHTLFKVSQCGAGGKGVIENFNKTVLKSLPRSLLLIEVESALMGAAFLSMLIFLLLCGAAYRPTEEIDLRSIGWGNIFQLPFKHLRDYRIRLLCPFFIYSGFEVLFAVQGLTLSYGVCAVGMEKLWLLVMVYGLSSSICSALALALLRLPRPVILLAGAFVHFVLIIALMCWSPTPRDTSRLPYLLVVSALWGLGTALNKTGLSTLLGMLYEDKERLDFVYTIYHWWQAIAIFIVYLWSALPMRAKLGLLLLTLLVASLCYVQMERRLAKKIPFRLPRIPRPRHKVKGYRYLEEENSDESDTDLSDADDDDDKEEDERMLVEEDRNTDGDSQGSPGPERKRVRDRRRKHDNTAYEQAGERDDYVQH

SEQ ID NO: 4 - protein, 568 aa, Columba livia
MEKDPNYLQEAAKGLAGDRLGAEGTEAQLDDFVGAQSDYNEEEEEQKYFRRKRLGIVKNVVAASLAGMLTYGVYLGLLQMQLILHYDETYREVKYSNIQLEDIDRKVLMGINVTPIAALLYTPVLIRFFGTKWAMFLAVGIYALFVSSNYWERYYTLVPSAVAIGVAIVPLWASMGNYIMRMAQKYYEYVNYKEDHVQEQQRPPRGACNTYIIVFQTVFYACFHLSFVCAQMPMLFFLNNYLYQLNHTLYNIKHCGTLSHGTLSGFNTTVLQSLPRSVNLIIVESALMAAAFLAMLTVLVLCGSAYQPMEEIDLRSIGWGNIFQLPFKHMRDYRLRHLFPLFIYSGFEELFVCTGFSLNYGVCALGLEKLAYLLMAYGFSASLCSSLALSMLRLRRQIPLLAGAFIHAALLVTLFCWAPQPRLVVQAPLLYTIAVLWGTGSALNKTSISILLGMLYEDKERQDFIFTIYHWWQALAIFTVYLWSELPMKAKLSIMLATLAVATGAYLWMEHKLAQHVAYRLPRIPRPRLRMHGYCYLEEDDSDETGSERRPTESASRDELPEDENQLG

SEQ ID NO: 5 - protein, 595 aa, Xenopus tropicalis
MADIYPEASINGIAADRGRNYAVQDDTPVLDGANAQVEDFLGEKPDYDEEAEEKRYYRRKRLLTVKSVVFASLGAALTYGVYLGLLQMQLILHYDETYRDVKYSNIGLQDIDSKMLMGINVTPIVALLYTPVLIRFFGTKWMMFLSSGIYALFVSTNYWERYYTLVPSATVIGVMIVPFWASLGNYITRMAQKYYEFVNYKEENVREQRRVPRGASHRYIIIFQSIFYSLFHISFVLAQLPVHFFLSKFFYSYNHTLFNVRDCGAAIQHGILPNFNLTVLHTLPPSLNLIYVESALMGAALVSMLLILLLCGSAYRPTEEIDLRSIGWGNIFQLPFKHARDYRLRCLAPYFLYSGFEVLFACTCFALCYGVCSLGLESLPFLLTAYGISASFSSSLALCMLRFPRQIPLVGGTLLHCCILIGLFFWSPTPKLQSQKPLLYLVAILWGVGSALNKSGLSIIIGVFYEDKERQDFVFTIYHWLQALAIFIVYLWSGIPVTAKLGIMLASALISVLSYLWMEHKLSCKVPYRVPKIPAPRHKVKGYRYMEEENSDESGSEGEKEQSEGGESEEEDKTVPCYRQPGRKECYEQAVGDEE

SEQ ID NO: 6 - protein, 570 aa, Callorhinchus milii
MENDRDATETGNVIRNSSPAEILEDRSTEIDDFIGPNLEYDAEIEEERHFWRKRVAVVKNLVCVSVACMLTYGIYLGLLQMQLILHYDETYREVKYGNMGLQDIDQKMLMGINVTPVVALLYTPVLIRFLGTKWLLFLCVGVYALFVSTNYWERYYTLVPSAIAIGFSIVPLWTSIGNYITRMAQKYYEYSNYKAIDVQQQKKAPKGAYEKYIVIFHSVFYAIFSLSLVCAELPMICFLTDYLYPYDHTLYNVKHCGPQMKGCLEGLNKTVLIKLPRSTSLIIVESVMMGVAFLSMLLVLMLCGAAYRPTEEIDLRSIGWGNIFQLPFKHLRDFRLRLLIPFFVFSGLEMVFACTGFALSYGVCSLGLERLSSLIMVYGLSWSLWSLLSLSLPRRGILVAGGVLHLTLLLTLMVWAPAPRKPTTGNMVTLHIIAGLWGLGTAFNKSGLSALLGILYSDKGRQDFIFAVYHWCQSMAIFGYYLLSNLQMQVKLSILLICLVISLLSFGWMERKIKRQVPYRIPRIPKPRHKTKGYRYLEEENSDEEEDGAEEKEMEQDGGEDERETLRRRD

SEQ ID NO: 7 - protein, 16 aa, Artificial Sequence
  Synthesized phospho-peptide
YLEEDNSDESDMEGEQ

SEQ ID NO: 8 - DNA, 20 bp, Artificial Sequence
  CpG-B
tccatgacgttcctgatgct

SEQ ID NO: 9 - DNA, 23 bp, Artificial Sequence
  Guide RNA
tgctgtggcttcggaatgcgcgg

SEQ ID NO: 10 - protein, 22 aa, Artificial Sequence
  3xFLAG peptide
DYKDHDGDYKDHDIDYKDDDDK

SEQ ID NO: 11 - protein, 9 aa, Artificial Sequence
  HA peptide
YPYDVPDYA

SEQ ID NO: 12 - DNA, 19 bp, Artificial Sequence
  Guide RNA
ctcacctacggcgtctacc

SEQ ID NO: 13 - DNA, 23 bp, Artificial Sequence
  Guide RNA
ggcgcttgcggcggtagtagcgg

SEQ ID NO: 14 - DNA, 23 bp, Artificial Sequence
  Guide RNA
cggagtggtcaagaacgtgctgg

SEQ ID NO: 15 - DNA, 23 bp, Artificial Sequence
  Guide RNA
ttcggaatgcgcggctgccgcgg

SEQ ID NO: 16 - DNA, 23 bp, Artificial Sequence
  Guide RNA
agtccgcggctaccgctacctgg

SEQ ID NO: 17 - DNA, 20 bp, Artificial Sequence
  Guide RNA
ggttcaagaacagcgatagg